(19) United States
(12) Patent Application Publication
(10) Pub. No.: US 2002/0034741 A1
(43) Pub. Date: Mar. 21, 2002

(54) Use of polypeptides or nucleic acids encoding these of the gene family NM23 for the diagnosis or treatment of skin or intestinal disorders, and their use for the identification of pharmacologically active substances

(76) Inventors: Sabine Werner, Zurich (CH); Susanne Braun, Zurich (CH); Jorn-Peter Halle, Penzberg (DE); Andreas Goppelt, Munchen (DE); Johannes Regenbogen, Martinsried (DE)

Correspondence Address: CLARK & ELBING LLP, 176 FEDERAL STREET, BOSTON, MA, 02110-2214, US

(21) Appl. No.: 09/791,118

(22) Filed: Feb. 22, 2001

Related U.S. Application Data
(60) Provisional application No. 60/199,312, filed on Apr. 24, 2000.

(30) Foreign Application Priority Data
Feb. 23, 2000 (DE) DE 100 08 330.7

(51) Int. Cl. C12Q 1/68; G01N 33/53; A61K 48/00; C07K 16/18

(52) U.S. Cl. 435/6; 435/7.1; 800/3; 514/44; 530/388.1

(57) ABSTRACT

Method of using of polypeptides or nucleic acids encoding these, of the gene family NM23, for the analysis and/or diagnosis and/or prevention and/or treatment of disorders of skin and/or intestinal disorders and/or wound healing and/or disorders of wound healing and/or for the identification of pharmacologically active substances.

### DESCRIPTION

The invention relates to the use of polypeptides or nucleic acids encoding these, of the gene family NM23, for the diagnosis and/or prevention and/or treatment of disorders of skin or intestinal cells and in wound healing, and/or disorders of wound healing and their use for the identification of pharmacologically active substances.

Wounds in general heal without therapeutic intervention. However, there are numerous disorders in which wound healing plays a role, such as, for example, diabetes mellitus, arterial occlusive diseases, psoriasis, Crohn's disease, epidermolysis bullosa, age-related skin changes or innervation disorders. Wound healing disorders lead to a delayed healing of wounds or to chronic wounds. These disorders can be caused by the nature of the wound (e.g. large-area wounds, deep and mechanically expanded operation wounds, burns, trauma, decubitus), medicinal treatment of the patients (e.g. with corticoids) but also by the nature of the disorder itself. For example, 25% of the patients with Type II diabetes thus frequently suffer from chronic ulcers (“diabetic foot”), of which approximately half necessitate expensive in-patient treatments and nevertheless finally heal poorly. Diabetic foot causes more stays in hospital than any other complication associated with diabetes. The number of these cases in diabetes Type I and II is on the increase and represents 2.5% of all hospital admissions. Moreover, wounds heal more poorly with increasing age of the patients. An acceleration of the natural wound healing process is often desirable as well in order to decrease, for example, the danger of bacterial infections or the rest periods of the patients.

Further disorders can also occur after successful wound closure. While foetal skin wounds heal without scar formation, formation of scars always occurs after injuries in the postnatal period, which often represents a great cosmetic problem. Moreover, the quality of life can be dramatically adversely affected in the case of patients with large-area burn wounds, especially as in scarred skin the appendages, such as hair follicles, sweat and sebaceous glands are missing. In the case of appropriate genetic disposition, keloids can also occur, hypertrophic scars which proliferate into the surrounding skin.

The process of skin healing requires complex actions and interactions of various cell types which proceed in a coordinated manner. In the wound healing process, the following steps are differentiated: clotting of blood in the area of the wound, the recruitment of inflammatory cells, reepithelialization, the formation of granulation tissue and the matrix remodeling. The exact reaction pattern of the cell types involved during the phases of proliferation, migration, matrix synthesis and contraction are, just like the regulation of genes such as, for example, growth factors, receptors and matrix proteins, little known up to now.

Thus until now only a few satisfactory therapies have been developed in order to be able to intervene in wound healing disorders. Established forms of therapy are restricted to physical assistance of wound healing (e.g. dressings, compresses, gels) or the transplantation of skin tissues, cultured skin cells and/or matrix proteins. In recent years, growth factors have been tested for improving wound healing without, however, improving the conventional therapy decisively. The diagnosis of wound healing disorders is also based on not very meaningful optical analysis of the skin, since a deeper understanding of the gene regulation during wound healing was lacking until now.

Not very satisfactory therapies have been developed until now for other disorders of regenerative processes of the skin and the intestine as well. Here too, the knowledge of gene regulation is advantageous for the development of diagnostics and therapies. It has been shown (Finch et al., 1997, Am. J. Pathol. 151: 1619-28; Werner, 1998, Cytokine Growth Factor Rev. 9: 153-165) that genes relevant to wound healing also play a crucial role in dermatological disorders which are based on disorders of the regeneration of the skin, and generally in regenerative processes. Thus the growth factor KGF not only plays a crucial role in the regulation of the proliferation and differentiation of keratinocytes during wound healing, but is also an important factor in the hyperproliferation of the keratinocytes in psoriasis and regeneration processes in the intestine (in Crohn's disease and colitis ulcerosa).

It is therefore the object of the present invention to make available polypeptides and/or nucleic acids encoding these which are involved in processes in disorders of skin or intestinal cells and/or wound healing and/or disorders of wound healing, and whose use decisively improves the diagnosis and/or prevention and/or treatment and also the identification and development of pharmaceuticals and/or diagnostics which are effective in connection with these disorders.

Disorders of the skin or the intestine, wound healing and disorders of wound healing are considered to be different from diseases associated with uncontrolled tissue growth and differentiation especially cancer of the skin and cancer of the intestine. In the latter type of diseases individual cells are transformed and start to proliferate in an uncontrolled, autonomous fashion, i.e. independent from interactions with other cell types, which transformed cells inherit their pathological changes to their daughter cells. In these diseases the loss of interactions is paralleled by a loss of cell-cell adhesion and typical cellular properties. In contrast, diseases according to the invention result from disorders of cellular interactions. The cause of skin diseases according to the invention depends on a variety of factors. For instances in the case of psoriasis a genetical predisposition as well as dysfunctional T-cells, fibroblasts and keratinocytes play a role (Nair et al., 1997; Hum. Molec. Genet. 6: 1349-1356; Gottlieb et al., 1995, Nat. Med. 1: 442-447; Saiag et al., 1985, Science, 230: 669-672; Pittelkow, 1998, in Roenigk 1998: 225-246). The course of wound healing can also be modulated by various endogenous and exogenous factors. Even small disturbances of the interactions of different cell types of the dermis and epidermis as well as interactions of these cell types with other tissues and organs such as the vascular system, the nervous system and the connective tissue can lead to severe disorders of wound healing followed by the formation of scars. Moreover, the process of wound healing can be affected by infections, ageing, vitamin deficiencies as well as diseases such as diabetes and disorders of the immune system. Similar complex interactions have been described for other disorders of the skin such as vitiligo and a atopic dermatitis. Based on such arguments skin diseases according to the invention can be distinguished from diseases associated with uncontrolled tissue growth and differentiation including cancer.

The autonomous character of cancerous diseases is also evident at the level of therapy. In the case of tumors that do not form metastases, the disease can be treated surgically. Such mechanical treatment is possible since tumor cells do not interact with adjacent cells or tissues. Therefore the patient can be healed by excision of the tumor whereas such treatment is not possible in the case of disorders of the skin according to the invention—the pathological disturbances of cell-cell and tissue-tissue interactions cannot be solved by simple excision of the affected parts of the skin.

By comparing the two different approaches of treatment for the two different types of diseases to be distinguished it is evident that two different mechanisms underlie the different diseases. In the case of diseases associated with uncontrolled tissue growth and differentiation especially cancer, the therapy is targeted to kill fast-growing cells, for example, by means of cytostatic agents. These toxic agents prevent the growth of actively proliferating cells, while cells of the G0-phase of the cell-cycle remain unaffected. In contrast, the treatment of disorders of the skin according to the invention is directed to modulate the cellular interactions between different types of cells, such as for example by influencing the migration, proliferation and differentiation of individual cell types. Disorders of the skin according to the invention cannot be treated by a general inactivation of proliferative cells.

The methodological approach to identify nucleic acids used according to the invention which are involved in wound healing and/or processes of disorders of skin or intestine according to the invention is very different from an approach which is suitable to identify nucleic acids involved in cancer. The latter could be identified by analysing differentially expressed genes in the type of cells affected by such diseases. The screening approach used in the invention aims at identifying genes involved in complex processes of disorders of the skin and/or wound healing and/or disorders of wound healing by comparing the gene expression of pathological and healthy tissue biopsies. Such an approach would not be suitable for the identification of genes involved in cancerous diseases.

The arguments raised to distinguish disorders of the skin according to the invention from skin diseases associated with uncontrolled tissue growth and differentiation especially skin cancer can also be applied in an analogous way to distinguish disorders of the intestine according to the invention from diseases of the intestine associated with uncontrolled tissue growth and differentiation especially cancer of the intestine. For instance, delayed healing of ulcers of the colon, e.g. Crohn's disease, as it has been shown for the skin, is caused and modulated by various factors which disturb the cellular interactions. Such factors include autoimmune mechanisms, cytokine polymorphisms, bacteria and infectious agents (Perner and Rask-Madsen, 1999, Aliment Pharmacol. Ther. 13: 135-144). These factors disturb the interactions between intestinal cells such as crypt cells, villus enterocytes or phagocytes (Ruemmele und Seidman, 1998, Chung Hua Min Kuo Hsiao Erh Ko I Hsueh Hui Tsa Chih, 39:1-8) The screen according to the invention is thus also not suitable to identify nucleic acids that are involved in cancer of the intestine-specific processes but the screen is powerful when applied to diseases of the intestine.

In the analysis of gene expression during the wound healing process as well as in psoriasis and Crohn's disease, it was possible to identify the gene family NM23 whose already known and described functions until now were not connected with skin or intestinal disorders, for example disturbed wound healing, but whose regulation is essential for the wound healing process and which are thus brought for the first time in a causal relationship with diagnosis and/or treatment of skin or intestinal disorders, for example disturbed wound healing. The polypeptides of these genes do not belong to the targets known until now for diagnosis—such as, for example, the indication—and/or the treatment—such as, for example, the modulation—of skin and/or intestinal disorders according to the invention and/or wound healing or for the identification of pharmacologically active substances for therapies of skin and/or intestinal disorders and/or wound healing, such that completely novel therapeutic approaches result from this invention.

The object is therefore achieved by the use of a polypeptide used according to the invention of the gene family NM23 according to one of SEQ ID No. 1 to SEQ ID No. 10 or functional variants thereof or nucleic acids encoding these or variants thereof for the diagnosis and/or treatment—for example for therapeutic and/or prophylactic treatment—of skin and/or intestinal disorders and/or wound healing and/or disorders of wound healing and/or for the identification of pharmacologically active substances.

The gene family NM23 codes for proteins having nucleotide diphosphate kinase (NDK) activity (reviewed in Postel, 1998, Int. J. Biochem. Cell. Biol. 30:1291-5), which convert nucleoside diphosphate into nucleoside triphosphate in a substrate unspecific manner. The active enzyme consists of 6 subunits of the highly homologous (see FIG. 6) polypeptides NM23A (also named NDKA, see FIG. 6) and NM23B (NDKB) and in which all 6 possible combinations of the subunits can occur (Gilles et al., 1991, J. Biol. Chem. 266:8784-9). The genes NM23-H1 (from human, EMBL database entries X17620, X75598, X73066; Rosengard et al., 1989, Nature 342:177-180) and NM23-M1 (from mouse, EMBL M35970, M65037, U85511, AF033377; Rosengard et al., supra; Steeg et al., 1988, J. Natl. Cancer Inst. 80:200-204), respectively, code for the polypeptide NM23A/NDKA (SWISSPROT database entries P15531 and P15532) while the genes NM23-H2 (EMBL X58965, M36981, L16785; Gilles et al., 1991, J. Biol. Chem. 266:8784-9; Stahl et al., 1991, Cancer Res. 51:445-9) and NM23-M2 (EMBL X68193; Urano et al., 1992, FEBS Lett. 309:358-362) code for NM23B/NDKB (SWISSPROT entries P22392 and Q01768).

Furthermore, the genes NM23-H4/NDKM (SWISSPROT entry 000746; Milon et al., 1997, Hum. Genet., 99:550-557); DR-NM23/NDK3 (SWISSPROT entry Q13232; Cucco et al., 1995, Proc. Natl. Acad. Sci. U.S.A., 92:7435-7439), NM23-H5/NDK5 (SWISSPROT entry P56597, Munier et al., 1998, FEBS Lett., 434:289-294), type 5 NM23 (EMBL entry U90449; Nakamura et al., 1997, direct entry into the database) and NDK6 (SWISSPROT entry 060361, Bradshaw and Ozersky, 1998, direct entry into the database) belong to the gene family NM23 as well. The polypeptides of the gene family NM23 show approx. 55 to 95% identity between each other on the amino acid sequence level.

Beside the function as a metabolic enzyme several other functions have been assigned to the gene family NM23. Thus, the gene products of NM 23B are not only located in the cytoplasm but also in the nucleus (Kraeft et al., 1996, Exp. Cell Res. 227:63-9), and a DNA binding as well as a transcriptional activation function was described (Postel et al., 1993, Science 261:478-80; Postel, 1999, J. Biol. Chem 274:22821-9). Furthermore, a role of NM23 in the regulation of RasGTPases was described (Zhu et al., Proc. Nat. Acad. Sci. USA 96:14911-8).

The reduced expression of NM23A was described as a tumor marker in the formation of metastases and cell aberrations in Drosophila melanogaster (Rosengard et al., 1989, Nature 342:177-180), wherein a normal level of expression of NM23 can inhibit the capability of tumor cells to form metastases (Lee and Lee, 1999, Cancer Letter 145:93-9). This function seems not to be linked to enzyme activity (Lee and Lee, supra). On the other hand the expression of NM23 is essential for cell proliferation, as can be assumed from antisense- (Cipollini et al., 1997, Int. J. Cancer 73:297-302) and antibody-experiments (Sorscher et al., 1993, Biochem. Biophys. Res. Commun. 195:336-45). Nevertheless, further analysis of the expression of NM23 in human early and metastases-forming melanoma cells as well as analysis of different stages of melanoma formation led to the result that the expression of NM23 in human, in contrast to the situation in the mouse, did not correlate with the formation of metastasis (Easty et al.; 1996, Br. J. Cancer 74(1): 109-14). Lately, these results could be confirmed during a second investigation by another group (Seregard et al., 1999, Exp. Eye Res. 69(6): 671-676). Therefore, NM23 seemed not to be suited for a reliable diagnosis in melanoma formation. Since the treatment of diseases associated with uncontrolled tissue growth and differentiation especially cancer differs greatly from the diseases according to the invention, it was therefore an unpromising strategy to use NM23 polypeptides and/or their functional variants and/or nucleic acids encoding these polypeptides for diagnosis or treatment of diseases according to the invention, as it has been described for the therapy in the context of diseases associated with uncontrolled tissue growth and differentiation especially cancer (WO 98/11232). Moreover no connection has been established between the polypeptides of the gene family NM23, nucleic acids or cDNAs encoding these polypeptides and disorders of the skin or the intestine or wound healing or disorders of wound healing. It was therefore unexpected that the nucleic acids and/or the polypeptides could be used according to the invention.

In general, the analysis of differentially expressed genes in tissues is afflicted with distinctly more errors in the form of false-positive clones than the analysis of cell culture systems. This cannot be circumvented by the use of a defined cell culture system, since existing culture systems are not able to simulate the complexity of the wound healing process satisfactorily.

The problem is particularly true for the skin which consists of a variety of different cell types. Moreover wound healing is a highly complex process involving spatial and temporal changes of cellular processes comprising proliferation and differentiation of various cell types. For the expert it is therefore an unpromising strategy to analyze not only the complex system of the skin but in addition the physiological process of wound healing and even different stages of wound healing at the level of differentially expressed genes. Based on these difficulties the success of the screening was dependent on the choice of experimental parameters. While the methods applied are standard (e. g. subtractive hybridization) the screening and verification strategy is inventive due to the sophisticated and defined choice of parameters. For instance the choice of the point of time of taking a biopsy sample is critical for the success of the screening: Disorders of wound healing and skin are often caused by disorders of the cellular proliferation and cellular migration. These processes are initiated one day after wounding. Therefore analysis of the molecular processes at this point of time would not provide much insight into the processes essential for normal wound healing. However, in the course of wound healing at points of time later than one day after wounding the composition of cell types in the wound has changed considerably. As a result the differential expression in this wound would not necessarily mean that the gene is differentially expressed in the cells, it might just reflect a different cell type composition. Therefore, the choice of the day to take biopsies is crucial for the success of the screening.

In spite of the defined parameters an over-representation of genes differentially expressed during wound healing was observed among the obtained genes, which are unsuitable for the use in wound healing or disorders of the skin. These genes comprise, for example, genes encoding for enzymes of the primary metabolism, such as glycolysis, citric-acid cycle, gluconeogenesis and respiratory chain, but also genes that code for ribosomal proteins, such as L41 and S20. Only a relatively small number of suitable genes could be identified. It was therefore surprising that the identified genes useable or according to the invention where genes relevant for wound healing.

In addition there are enormous variabilities of the condition of the wound at the time when the biopsy is taken from potential patients visiting the physician for the first time. An animal model was therefore used for the identification of the nucleic acids used according to the invention. BALB/c mice were wounded and wound biopsies were taken at various points in time. This process has the advantage that the boundary conditions such as genetic background, type of the wound, point of time when taking biopsies etc. can be controlled exactly and thus allow a reproducible analysis of gene expression. Even under the defined conditions of the animal model further methodological problems occur such as redundancy of the analyzed clones and underrepresentation of weakly expressed genes, which complicate the identification of relevant genes.

The accession numbers of the polypeptides of the gene family NM23 according to the invention and their cDNAs are shown in FIG. 5. The cDNAs of the polypeptides NM23-M2 useable according to the invention were isolated from cDNA libraries obtained from intact and wounded skin. cDNAs were selected, which showed different abundance in the cDNA libraries of normally healing vs. badly healing (Dexamethasone treated) wounds (example 1). This was done by means of subtractive hybridization (Diatchenko et al., 1996, Proc. Nat. Acad. Sci. USA 93: 6025-6030). The selected cDNA showed a higher abundance in the cDNA pool of Dexamethason-treated animals relative to the cDNA pool of normally healing wounds. NM23-M2 could be identified through a subtractive hybridization of mouse wound cDNA. NM23-M2 was enriched both in a cDNA population which was obtained from a subtraction of normally healing 1-day wounds against intact skin and in a cDNA population which was obtained from the subtraction of badly healing wounds (dexamethasone treated mice) against well healing wounds (example 1). This suggested that NM23 is not only regulated during normal wound healing, but that the regulation of the expression is essential for the normal progression of wound healing.

After the initial identification of a gene, it is necessary to confirm the wound healing-specific expression by another method. This was performed by so-called “reverse Northern blots”, “RNase protection assays”, “RT-PCR assays”,“in situ hybridization” and “TaqMan analysis”. Using these methods, the amount of mRNA in biopsies taken at various wound healing states and from skin disorders (psoriasis) and from intestinal disorders (Crohn's disease) was measured. Thereby, tissue-specific local changes of the expression pattern in skin and intestinal biopsies were determined (example 2-7).

In the reversed Northern blot the enrichment of the NM23-M2 cDNA after subtraction could be confirmed (FIG. 1, example 1). Furthermore, in situ hybridizations on tissue-slices of 5-day wounds of mice showed that the gene NM23-M1 was expressed in the hyperproliferative epithelium at the wound edge, which argues in favor of a crucial role in the proliferation of keratinocytes and reepitheliazation of the wound (example 4). It could also be shown by quantitative RT-PCR analysis that the expression of NM23-M2 was approx. 5 times stronger in poorly healing wounds of dexamethasone treated mice than in normal well-healing wounds of control animals (FIG. 2, example 2). Thus, the essential role of NM23 in the wound healing process suggested by the subtraction experiments could be confirmed.

Furthermore, the expression of NM23 could be linked to psoriasis. An “RNase protection assay” was performed using RNA obtained either from biopsies of lesional skin areas of psoriasis patients or from biopsies of control subjects with normal skin, respectively. It could be shown that NM23-H1 was significantly stronger expressed in the skin of patients in comparison to skin of control subjects (FIG. 3, example 3). Thus, also in the case of psoriasis there is a connection between NM23 and the progression of the disease.

A similar correlation was found in the case of the inflammatory intestinal disorder Crohn's disease. Intestinal biopsies were taken from patients from clearly and less inflamed areas. In comparison to an intestine biopsy of a healthy control subject, all intestine biopsies of the Crohn's disease patients showed a significantly increased expression of NM23-H1 (FIG. 4, example 3). Furthermore, a correlation of the NM23-H1 expression with the severity of the disease could be found. Whilst less inflamed areas of the intestine showed only a moderate increase in the expression of NM23-H1, a strong increase in the expression could be found for clearly inflamed areas (FIG. 4). These results demonstrate that the expression of NM23 not only reflects the severity of the disease, but is essential for the progression of the disease and that the expression of NM23 can be used as a diagnostic marker for these diseases.

The polypeptides used according to the invention can furthermore be characterized in that they are synthetically prepared. Thus, the entire polypeptide or parts thereof can be synthesized, for example, with the aid of the conventional synthesis (Merrifield technique). Parts of the polypeptides used according to the invention are particularly suitable for the obtainment of antisera, which can be used to search suitable gene expression banks to arrive at further functional variants of the polypeptides used according to the invention.

The term “functional variants” within the meaning of the present invention is understood as meaning polypeptides which are functionally related to the polypeptides useable according to the invention, i.e. are regulated during regenerative processes of the skin or the intestine and/or have structural features of the polypeptides. Examples of functional variants are polypeptides which are encoded in various individuals or in various organs of an organism by different alleles of the gene.

Other examples include, for example, polypeptides which are encoded by a nucleic acid, which are isolated from non-skin or non-intestine-specific tissue, e.g. embryonic tissue, but after expression have the indicated functions in a cell involved in wound healing.

In a further sense, this term is also understood as meaning polypeptides which have a sequence homology, in particular a sequence identity, of about 70%, preferably about 80%, in particular about 90%, especially about 95%, to the polypeptide having the amino acid sequence according to one of SEQ ID No. 1 to SEQ ID No. 10 and/or with the aid of the DNA sequences to the publicly accessible database entries of the list in FIG. 5.

Functional variants also comprise parts of polypeptides useable according to the invention with a length of at least 6 amino acids, preferentially at least 8 amino acids, especially preferred with at least 12 amino acids.

In addition, these also include N- and/or C-terminal and/or internal deletions or parts of the polypeptide in the range from about 1-60, preferably from about 1-30, in particular from about 1-15, especially from about 1-5 amino acids. For example, the first amino acid methionine can be absent without the function of the polypeptide being significantly altered.

The term “coding nucleic acid” relates to a DNA sequence which codes for an isolatable polypeptide useable according to the invention or a precursor e.g. containing a signal sequence. The polypeptide can be encoded by a sequence of full length or any part of the coding sequence as long as the specific, for example enzymatic, activity is retained.

It is known that alterations in the sequence of the nucleic acids used according to the invention can be present, for example, due to the degeneration of the genetic code, or that untranslated sequences can be attached to the 5′ and/or 3′ end of the nucleic acid without its activity being significantly altered; modifications mentioned below can also be applied to nucleic acids. This invention therefore also comprises so-called “variants” of the nucleic acids used according to the invention.

The term “variants” indicates all DNA sequences which are complementary to a DNA sequence, which hybridize with the reference sequence under stringent conditions and have an overall similar activity to the corresponding polypeptide used according to the invention.

The term “regulation” is understood as meaning an increase or decrease in the amount of polypeptide or nucleic acids encoding for these polypeptides, such changes occur, for example, at the level of transcription or translation.

“Stringent hybridization conditions” are understood as meaning those conditions in which hybridization takes place at, for example, 60° C. in 2.5× SSC buffer, followed by a number of washing steps at 37° C. in a lower buffer concentration, and remains stable.

Variants of nucleic acids also include parts of nucleic acids useable according to the invention with a length of at least 8 nucleotides, preferentially at least 18 nucleotides, especially at least 24 nucleotides, especially preferred with at least 30 nucleotides, particularly preferred with at least 42 nucleotides.

Preferentially, the nucleic acids used according to the invention are DNA or RNA, preferably a DNA, in particular a double-stranded DNA. The sequence of the nucleic acids can furthermore be characterized in that it has at least one intron and/or one polyA sequence. The nucleic acids used according to the invention can also be used in the form of their antisense sequence.

For the expression of a gene used according to the invention, in general a double-stranded DNA is preferred, the DNA region coding for the polypeptide being particularly preferred. This region begins with the first start codon (ATG) lying in a Kozak sequence (Kozak, 1987, Nucleic. Acids Res. 15: 8125-48) up to the next stop codon (TAG, TGA or TAA), which lies in the same reading frame to the ATG.

A further use of the nucleic acid sequences used according to the invention is the construction of anti-sense oligonucleotides (Zheng and Kemeny, 1995, Clin. Exp. Immunol. 100: 380-2; Nellen and Lichtenstein, 1993, Trends Biochem. Sci. 18: 419-23; Stein, 1992, Leukemia 6: 967-74) and/or ribozymes (Amarzguioui, et al. 1998, Cell. Mol. Life Sci. 54: 1175-202; Vaish, et al., 1998, Nucleic Acids Res. 26: 5237-42; Persidis, 1997, Nat. Biotechnol. 15: 921-2; Couture and Stinchcomb, 1996, Trends Genet. 12: 510-5). Using antisense oligonucleotides, the stability of the nucleic acid used according to the invention can be decreased and/or the translation of the nucleic acid used according to the invention inhibited. Thus, for example, the expression of the corresponding genes in cells can be decreased both in vivo and in vitro. Oligonucleotides or ribozymes can therefore be suitable as therapeutics. This strategy is suitable, for example, even for skin, epidermal and dermal cells, in particular if the antisense oligonucleotides are complexed with liposomes (Smyth et al., 1997, J. Invest. Dermatol. 108: 523-6; White et al., 1999, J. Invest. Dermatol. 112: 699-705; White et al., 1999, J. Invest. Dermatol. 112: 887-92). For use as a probe or as an “antisense” oligonucleotide, a single-stranded DNA or RNA is preferred.

Furthermore, a nucleic acid which has been prepared synthetically can be used for carrying out the invention. Thus, the nucleic acid used according to the invention can be synthesized, for example, chemically with the aid of the DNA sequences described in FIG. 5 and/or with the aid of the protein sequences likewise described in these figures with reference to the genetic code, e.g. according to the phosphotriester method (see, for example, Uhlmann, E. & Peyman, A. (1990) Chemical Reviews, 90, 543-584, No. 4).

Generally, oligonucleotides are rapidly degraded by endo- or exonucleases, in particular by DNases and RNases occurring in the cell. It is therefore advantageous to modify the nucleic acid in order to stabilize it against degradation, so that a high concentration of the nucleic acid is maintained in the cell over a long period (Beigelman et al., 1995, Nucleic Acids Res. 23: 3989-94; Dudycz, 1995, WO9511910; Macadam et al., 1998, WO9837240; Reese et al., 1997, WO9729116) . Typically, such a stabilization can be obtained by the introduction of one or more internucleotide phosphorus groups or by the introduction of one or more non-phosphorus internucleotides.

Suitable modified internucleotides are summarized in Uhlmann and Peymann (1990 Chem. Rev. 90, 544) (see also Beigelman et al., 1995 Nucleic Acids Res. 23: 3989-94; Dudycz, 1995, WO 95/11910; Madadam et al., 1998, WO 98/37240; Reese et al., 1997, WO 97/29116). Modified internucleotide phosphate radicals and/or non-phosphorus bridges in a nucleic acid which can be employed in one of the uses according to the invention comprise, for example, methylphosphonate, phosphorothioate, phosphoramidate, phosphorodithioate, phosphate ester, while non-phosphorus internucleotide analogues, for example, contain siloxane bridges, carbonate bridges, carboxymethyl esters, acetamidate bridges and/or thioether bridges. It is also intended that this modification should improve the shelf life of a pharmaceutical composition which can be employed in one of the uses according to the invention.

In a further embodiment of the invention, the nucleic acids used according to the invention are used for the preparation of a vector, preferably in the form of a shuttle vector, phagemid, cosmid, expression vector or vector having gene therapy activity to be used for analysis and/or diagnosis and/or prevention and/or treatment of disorders of skin and/or intestine and/or wound healing and/or disorders of wound healing. Furthermore, knock-out gene constructs or expression cassettes can be prepared using the nucleic acids.

Thus, the nucleic acid used according to the invention can be contained in a vector, preferably in an expression vector or vector suitable for gene therapy. Preferably, the vector suitable for gene therapy contains wound- intestine- or skin-specific regulatory sequences which are functionally associated with the nucleic acid useable according to the invention.

The expression vectors can be prokaryotic or eukaryotic expression vectors. Examples of prokaryotic expression vectors are, for expression in E. coli, e.g. the vectors pGEM or pUC derivatives, examples of eukaryotic expression vectors are for expression in Saccharomyces cerevisiae, e.g. the vectors p426Met25 or 426GAL1 (Mumberg et al. (1994) Nucl. Acids Res., 22, 5767-5768), for expression in insect cells, e.g. Baculovirus vectors such as disclosed in EP-B1-0 127 839 or EP-B1-0 549 721, and for expression in mammalian cells, e.g. the vectors Rc/CMV and Rc/RSV or SV40 vectors, which are all generally obtainable.

In general, the expression vectors also contain promoters suitable for the respective host cell, such as, for example, the trp promoter for expression in E. coli (see, for example, EP-B1-0 154 133), the Met 25, GAL 1 or ADH2 promoter for expression in yeasts (Russel et al. (1983), J. Biol. Chem. 258, 2674-2682; Mumberg, supra), the Baculovirus polyhedrin promoter, for expression in insect cells (see, for example, EP-B1-0 127 839). For expression in mammalian cells, for example, suitable promoters are those which allow a constitutive, regulatable, tissue-specific, cell-cycle-specific or metabolism-specific expression in eukaryotic cells. Regulatable elements according to the present invention are promoters, activator sequences, enhancers, silencers and/or repressor sequences.

Examples of suitable regulatable elements which make possible constitutive expression in eukaryotes are promoters which are recognized by the RNA polymerase III or viral promoters, CMV enhancer, CMV promoter, SV40 promoter or LTR promoters, e.g. from MMTV (mouse mammary tumor virus; Lee et al. (1981) Nature 214, 228-232) and further viral promoter and activator sequences, derived from, for example, HBV, HCV, HSV, HPV, EBV, HTLV or HIV.

Examples of regulatable elements which allow regulatable expression in eukaryotes are the tetracycline operator in combination with a corresponding repressor (Gossen M. et al. (1994) Curr. Opin. Biotechnol. 5, 516-20).

Preferably, the expression of wound-healing-relevant genes takes place under the control of tissue-specific promoters, wherein wound-, skin- or intestine-specific promoters such as, for example, the human K10-promoter (Bailleul et al., 1990. Cell 62: 697-708), the human K14-promoter (Vassar et al., 1989, Proc. Natl. Acad. Sci. USA 86: 1563-67), the bovine cytokeratin IV- promoter (Fuchs et al., 1988; The biology of wool and hair (ed. G. E. Rogers, et al.), pp. 287-309. Chapman and Hall, London/New York) or the “fatty acid binding protein” promoter from the rat are particularly to be preferred.

Further examples of regulatable elements which allow tissue-specific expression in eukaryotes are promoters or activator sequences from promoters or enhancers of those genes which code for proteins which are only expressed in certain cell types.

Examples of regulatable elements whichallow cell cycle-specific expression in eukaryotes are promoters of the following genes: cdc25, cyclin A, cyclin E, cdc2, E2F, B-myb or DHFR (Zwicker J. and Müller R. (1997) Trends Genet. 13, 3-6).

Examples of regulatable elements which make possible metabolism-specific expression in eukaryotes are promoters which are regulated by hypoxia, by glucose deficiency, by phosphate concentration or by heat shock.

In order to make possible the introduction of nucleic acids used according to the invention and thus the expression of the polypeptide in a eu- or prokaryotic cell by transfection, transformation or infection, the nucleic acid can be present as a plasmid, as part of a viral or non-viral vector. Suitable viral vectors are particularly: baculoviruses, vaccinia viruses, adenoviruses, adeno-associated viruses and herpesviruses. Suitable non-viral vectors are particularly: virosomes, liposomes, cationic lipids, or poly-lysine-conjugated DNA.

Examples of vectors suitable for gene therapy are virus vectors, for example adenovirus vectors or retroviral vectors (Lindemann et al., 1997, Mol. Med. 3: 466-76; Springer et al., 1998, Mol. Cell. 2: 549-58). Eukaryotic expression vectors are suitable in isolated form for use in gene therapy, as naked DNA can penetrate into skin cells after topical application (Hengge et al., 1996, J. Clin. Invest. 97: 2911-6; Yu et al., 1999, J. Invest. Dermatol. 112: 370-5).

Vectors suitable for gene therapy can also be obtained by complexing the nucleic acid used according to the invention with liposomes, since a very high transfection efficiency, in particular of skin cells, can thus be achieved (Alexander and Akhurst, 1995, Hum. Mol. Genet. 4: 2279-85). In the case of lipofection, small unilamellar vesicles are prepared from cationic lipids by ultrasonic treatment of the liposome suspension. The DNA is bound ionically to the surface of the liposomes, namely in such a ratio that a positive net charge remains and the plasmid DNA is complexed to 100% by the liposomes. In addition to the lipid mixtures DOTMA (1,2-dioleyloxypropyl-3-trimethylammonium bromide) and DPOE (dioleoylphosphatidylethanolamine) employed by Felgner et al. (1987, supra), numerous novel lipid formulations were synthesized meanwhile and tested for their efficiency in the transfection of various cell lines (Behr, J. P. et al. (1989), Proc. Natl. Acad. Sci. USA 86, 6982-6986; Felgner, J. H. et al. (1994) J. Biol. Chem. 269, 2550-2561; Gao, X. & Huang, L. (1991), Biochim. Biophys. Acta 1189, 195-203). Examples of the novel lipid formulations are DOTAP N-[1-(2,3-dioleoyloxy)propyl]-N,N,N-trimethylammonium ethyl-sulphate or DOGS (TRANSFECTAM; dioctadecylamidoglycylspermine). Auxiliaries which increase the transfer of nucleic acids into the cell can be, for example, proteins or peptides which are bound to DNA or synthetic peptide-DNA molecules which allow the transport of the nucleic acid into the nucleus of the cell (Schwartz et al. (1999) Gene Therapy 6, 282; Brandén et al. (1999) Nature Biotech. 17, 784). Auxiliaries also include molecules which allow the release of nucleic acids into the cytoplasm of the cell (Planck et al. (1994) J. Biol. Chem. 269, 12918; Kichler et al. (1997) Bioconj. Chem. 8, 213) or, for example, liposomes (Uhlmann and Peymann (1990) supra). Another particularly suitable form of gene therapy vectors can be obtained by applying the nucleic acid useable according to the invention to gold particles and shooting these into tissue, preferably into the skin, or cells with the aid of the so-called gene gun (Wang et al., 1999, J. Invest. Dermatol., 112: 775-81, Tuting et al., 1998, J. Invest. Dermatol. 111: 183-8).

A further form of a vector suitable for gene therapy useable according to the invention can be prepared by the introduction of “naked” expression vectors into a biocompatible matrix, for example a collagen matrix. This matrix can be introduced into wounds in order to transfect the immigrating cells with the expression vector and to express the polypeptides used according to the invention in the cells (Goldstein and Banadio, U.S. Pat. No. 5,962,427).

For the use of the nucleic acid useable according to the invention in gene therapy, it is also advantageous if the part of the nucleic acid which codes for the polypeptide contains one or more non-coding sequences including intron sequences, preferably between promoter and the start codon of the polypeptide, and/or a polyA sequence, in particular the naturally occurring polyA sequence or an SV40 virus polyA sequence, especially at the 3′ end of the gene, as a stabilization of the mRNA can be achieved thereby (Jackson, R. J. (1993) Cell 74, 9-14 and Palmiter, R. D. et al. (1991) Proc. Natl. Acad. Sci. USA 88, 478-482).

Knock-out gene constructs are known to the person skilled in the art, for example, from the U.S. Pat No. 5,625,122; U.S. Pat. No. 5,698,765; U.S. Pat. No. 5,583,278 and U.S. Pat. No. 5,750,825.

The present invention further relates to the use of a host cell, in particular a skin or intestinal cell, which is transformed using a vector useable according to the invention or a knock-out gene construct to be used for analysis and/or diagnosis and/or prevention and/or treatment of disorders of skin and/or intestine and/or wound healing and/or disorders of wound healing. Host cells can be either prokaryotic or eukaryotic cells, examples of prokaryotic host cells are E. coli and examples of eukaryotic cells are Saccharomyces cerevisiae or insect cells.

A particularly preferred transformed host cell useable according to the invention is a transgenic embryonic non-human stem cell, which is characterized in that it comprises a knock-out gene construct useable according to the invention or an expression cassette useable according to the invention. Processes for the transformation of host cells and/or stem cells are well known to the person skilled in the art and include, for example, electroporation or microinjection.

The invention further relates to a transgenic non-human mammal whose genome comprises a knock-out gene construct useable according to the invention or an expression cassette useable according to the invention to be used for analysis and/or diagnosis of disorders of skin and/or intestine and/or wound healing and/or disorders of wound healing. Transgenic animals show depending on the promoter used a tissue-specific, generally an increased expression of the nucleic acids and/or polypeptides and can be used for the analysis of wound healing disorders. Thus, for example, an activin A transgenic mouse exhibits improved wound healing (Munz et al., 1999, EMBO J. 18: 5205-15) while a transgenic mouse having a dominantly negative KGF receptor exhibits delayed wound healing (Werner et al., 1994, Science 266: 819-22). Moreover, transgenic animals could be equipped with accelerated wound healing abilities.

Processes for the preparation of transgenic animals, in particular of the mouse, are likewise known to the person skilled in the art from DE 196 25 049 and U.S. Pat. No. 4,736,866; U.S. Pat. No. 5,625,122; U.S. Pat. No. 5,698,765; U.S. Pat. No. 5,583,278 and U.S. Pat. No. 5,750,825 and include transgenic animals which can be produced, for example, by means of direct injection of expression vectors (see above) into embryos or spermatocytes or by means of the transfection of expression vectors into embryonic stem cells (Polites and Pinkert: DNA Microinjection and Transgenic Animal Production, page 15 to 68 in Pinkert, 1994: Transgenic animal technology: a laboratory handbook, Academic Press, London, UK; Houdebine, 1997, Harwood Academic Publishers, Amsterdam, The Netherlands; Doetschman: Gene Transfer in Embryonic Stem Cells, page 115 to 146 in Pinkert, 1994, supra; Wood: Retrovirus-Mediated Gene Transfer, page 147 to 176 in Pinkert, 1994, supra; Monastersky: Gene Transfer Technology; Alternative Techniques and Applications, page 177 to 220 in Pinkert, 1994, supra).

If nucleic acids used according to the invention are integrated into so-called targeting vectors (Pinkert, 1994, supra), it is possible after transfection of embryonic stem cells and homologous recombination, for example, to generate knock-out mice which, in general, as heterozygous mice, show decreased expression of the nucleic acid, while homozygous mice no longer exhibit expression of the nucleic acid. The animals thus produced can also be used for the analysis of wound healing disorders. Thus, for example, the eNOS (Lee et al., 1999, Am. J. Physiol. 277: H1600-1608), Nf-1 (Atit et al., 1999, J. Invest. Dermatol. 112: 835-42) and osteopontin (Liaw et al., 1998, J. Clin. Invest. 101: 967-71) knock-out mice exhibit impaired wound healing. Here too, a tissue-specific reduction of the expression of wound healing-relevant genes, for example in skin-specific cells using the Cre-loxP system (stat3 knock-out, Sano et al., EMBO J 1999 18: 4657-68), is particularly to be preferred. Transgenic and knock-out cells or animals produced in this way can also be used for the screening and for the identification of pharmacologically active substances or vectors suitable for gene therapy, respectively.

The invention further relates to the use of at least one polypeptide useable according to the invention and/or at least one nucleic acid coding for above mentioned polypeptides for analysis and/or diagnosis and/or prevention and/or treatment of disorders of skin and/or intestine and/or wound healing and/or disorders of wound healing and/or for the identification of pharmacologically active substances in a suitable host cell.

The polypeptide is prepared, for example, by expression of the nucleic acid used according to the invention in a suitable expression system, as already described above, used according to the methods generally known to the person skilled in the art. Suitable host cells are, for example, the E. coli strains DHS, HB101 or BL21, the yeast strain Saccharomyces cerevisiae, the insect cell line Lepidoptera, e.g. from Spodoptera frugiperda, or the animal cells COS, Vero, 293, HaCaT, and HeLa, which are all generally obtainable.

The invention further relates to the use of fusion proteins, which are produced by expression of nucleic acids according to the invention in a suitable host cell for analysis and/or diagnosis and/or prevention and/or treatment of disorders of skin and/or intestine and/or wound healing and/or disorders of wound healing or for the identification of pharmacologically active substances in a suitable host cell. The fusion proteins either have already the function of a polypeptide used according to the invention or are functionally active only after cleavage of the fusion portion. Especially included here are fusion proteins having about 1-200, preferably about 1-150, in particular about 1-100, especially about 1-50, foreign amino acids. Examples of such peptide sequences are prokaryotic peptide sequences, which can be derived, for example, from the galactosidase of E. coli. Furthermore, viral peptide sequences, such as, for example, of the bacteriophage M13 can also be used in order thus to produce fusion proteins for the phage display process known to the person skilled in the art.

Additional preferred examples of peptide sequences to be used for fusion proteins are peptides, which facilitate the detection of the fusion protein, for example “green fluorescent protein” (WO 95/07463) or functional variants thereof.

For the purification of the proteins according to the invention (a) further polypeptide(s) (tag) can be attached. Protein tags according to the invention allow, for example, high-affinity absorption to a matrix, stringent washing with suitable buffers without eluting the complex to a noticeable extent and subsequently selective elution of the absorbed complex. Examples of the protein tags known to the person skilled in the art are a (His)₆ tag, a Myc tag, a FLAG tag, a Strep tag, a Strep tag II, a haemagglutinin tag, glutathione transferase (GST) tag, intein having an affinity chitin-binding tag or maltose-binding protein (MBP) tag. These protein tags can be located N- or C-terminally and/or internally.

The invention further relates to the use of an antibody, preferably a polyclonal or monoclonal antibody to be used for analysis and/or diagnosis and/or prevention and/or treatment of disorders of skin and/or intestine and/or wound healing and/or disorders of wound healing and/or for the identification of pharmacologically active substances

Thus, for example, the local injection of monoclonal antibodies against TGF beta 1 can improve wound healing in the animal model (Ernst et al., 1996, Gut 39: 172-5).

In order to produce an antibody a polypeptide used according to the invention or functional variants thereof or parts thereof with at least 6 amino acids, preferentially at least 8 amino acids, especially preferred with at least 12 amino acids is used.

The process is carried out according to methods generally known to the person skilled in the art by immunizing a mammal, for example a rabbit, with the polypeptide used according to the invention or the mentioned parts thereof, if appropriate in the presence of, for example, Freund's adjuvant and/or aluminium hydroxide gels (see, for example, Diamond, B. A. et al. (1981) The New England Journal of Medicine, 1344-1349). The polyclonal antibodies raised in the animal as a result of an immunological reaction can then be easily isolated from the blood according to generally known methods and purified, for example, by means of column chromatography. Monoclonal antibodies can be produced, for example, according to the known method of Winter & Milstein (Winter, G. & Milstein, C. (1991) Nature, 349, 293-299).

As an alternative to classical antibodies it is also possible to utilize lipocaline based so-called “anticalines” (Beste et al., 1999, Proc. Natl. Acad. Sci. USA, 96:1898-1903). The natural ligand-binding-sites of lipocaline, such as for example retinol-binding protein or biline-binding protein, which, for example, can be modified by means of “combinatorial protein design approach” in such a way that the selected haptens bind, for example, polypeptides used according to the invention (Skerra, 2000, Biochim. Biophys. Acta 1482:337-350). Further known “scaffolds” which can be used as an alternative to antibodies have been described (Skerra, J. Mol. Recognit., 2000, 13:167-187).

The antibody used according to the invention is directed against a polypeptide used according to the invention and reacts specifically with the polypeptides used according to the invention, where the above-mentioned parts of the polypeptide are either immunogenic themselves or can be rendered immunogenic by coupling to suitable carriers, such as, for example, bovine serum albumin, or can be increased in their immunogenicity. This antibody is either polyclonal or monoclonal, preferably it is a monoclonal antibody. The term antibody is understood according to the present invention as also meaning antibodies or antigen-binding parts thereof prepared by genetic engineering and optionally modified, such as, for example, chimeric antibodies, humanized antibodies, multifunctional antibodies, bi- or oligospecific antibodies, single-stranded antibodies, F(ab) or F(ab)₂ fragments (see, for example, EP-B1-0 368 684, U.S. Pat. No. 4,816,567, U.S. Pat. No. 4,816,397, WO 88/01649, WO 93/06213, WO 98/24884).

The present invention furthermore relates to the use of a medicament comprising at least one nucleic acid used according to the invention, at least one polypeptide used according to the invention or at least one antibody used according to the invention, if appropriate combined together with suitable additives and auxiliaries to be used for analysis and/or diagnosis and/or prevention and/or treatment of disorders of skin and/or intestine and/or wound healing and/or disorders of wound healing.

The therapy of the disorders, in particular skin or intestinal disorders and/or of wound healing and/or disorders of wound healing can be carried out in a conventional manner, e.g. by means of dressings, plasters, compresses or gels which contain the medicaments useable according to the invention. It is thus possible to administer the pharmaceuticals containing the suitable additives or auxiliaries, such as, for example, physiological saline solution, demineralized water, stabilizers, proteinase inhibitors, gel formulations, such as, for example, white petroleum jelly, highly liquid paraffin and/or yellow wax, etc., topically and locally in order to influence wound healing immediately and directly. The administration of the medicaments used according to the invention can furthermore also be carried out topically and locally in the area of the wound, if appropriate in the form of liposome complexes or gold particle complexes. Furthermore, the treatment can be carried out by means of a transdermal therapeutic system (TTS), which makes possible a temporally controlled release of the medicaments useable according to the invention. The treatment by means of the medicaments useable according to the invention, however, can also be carried out by means of oral dosage forms, such as, for example, tablets or capsules, by means of the mucous membranes, for example the nose or the oral cavity, or in the form of dispositories implanted under the skin. TTS are known for example, from EP 0 944 398 A1, EP 0 916 336 A1, EP 0 889 723 A1 or EP 0 852 493 A1.

For gene therapy use in man, a medicament is especially suitable which contains the nucleic acid used according to the invention in naked form or in the form of one of the vectors suitable for gene therapy described above or in a form complexed with liposomes or gold particles. The pharmaceutical vehicle is, for example, a physiological buffer solution, preferably having a pH of about 6.0-8.0, especially of about 6.8-7.8, in particular of about 7.4, and/or an osmolarity of about 200-400 milliosmol/liter, preferably of about 290-310 milliosmol/liter. In addition, the pharmaceutical vehicle can contain suitable stabilizers, such as, for example, nuclease inhibitors, preferably complexing agents such as EDTA and/or other auxiliaries known to the person skilled in the art.

The administration of the nucleic acid used according to the invention, if appropriate in the form of the virus vectors described in greater detail above or as liposome complexes or gold particle complex usually takes place topically and/or locally in the area of the wound. It is also possible to administer the polypeptide itself with suitable additives or auxiliaries, such as, for example, physiological saline solution, demineralized water, stabilizers, proteinase inhibitors, gel formulations, such as, for example, white petroleum jelly, highly liquid paraffin and/or yellow wax, etc., in order to influence the wound healing immediately and directly.

The present invention furthermore relates to the use of a diagnostic for the diagnosis of disorders of the skin and/or intestinal disorders and/or wound healing and/or disorders in wound healing, which comprises at least one nucleic acid, at least one polypeptide or at least one antibody useable according to the invention, if appropriate together with suitable additives and auxiliaries.

For example, it is possible to prepare a diagnostic useable according to the present invention based on the polymerase chain reaction (Example 2, PCR diagnostic, e.g. according to EP 0 200 362) or an RNase protection assay, such as shown in detail in Example 3, with the aid of a nucleic acid used according to the invention. These tests are based on the specific hybridization of the nucleic acids useable according to the invention with the complementary counter strand, usually of the corresponding mRNA. The nucleic acid used according to the invention can in this case also be modified, such as described, for example, in EP 0 063 879. Preferably a DNA fragment used according to the invention is labelled according to generally known methods by means of suitable reagents, e.g. radioactively with α-³²P-dCTP or non-radioactively with biotin or digoxigenin, and incubated with isolated RNA, which has preferably been bound beforehand to suitable membranes of, for example, cellulose or nylon. With the same amount of investigated RNA from each tissue sample, the amount of mRNA which was specifically labelled by the probe can thus be determined. Alternatively, the determination of mRNA can also be carried out in tissue slices with the aid of in situ hybridization (see example 4 and Werner et al., 1992, Proc. Natl. Acad. Sci. USA 89: 6896-6900).

With the aid of the diagnostic useable according to the invention, a tissue sample can thus also be specifically measured in vitro for the strength of expression of the corresponding gene in order to be able to safely diagnose a possible wound healing disorder, intestinal disorders or dermatological disorders (Examples 1 to 3, 5, 6). Such a process is particularly suitable, for example, for the early prognosis of disorders.

A further diagnostic useable according to the invention contains the polypeptide used according to the invention or the immunogenic parts thereof described in greater detail above. The polypeptide or the parts thereof, which are preferably bound to a solid phase, e.g. of nitrocellulose or nylon, can be brought into contact in vitro, for example, with the body fluid to be investigated, e.g. wound secretion, in order thus to be able to react, for example, with autoimmune antibodies. The antibody-peptide complex can then be detected, for example, with the aid of labelled antihuman IgG or antihuman IgM antibodies. The labelling involves, for example, an enzyme, such as peroxidase, which catalyses a color reaction. The presence and the amount of autoimmune antibody present can thus be detected easily and rapidly by means of the colour reaction.

Another diagnostic contains the antibodies used according to the invention themselves. With the aid of these antibodies, it is possible, for example, to easily and rapidly investigate a tissue sample as to whether the concerned polypeptide is present in an increased amount in order to thereby obtain an indication of a possible wound healing disorder. In this case, the antibodies used according to the invention are labelled, for example, with an enzyme, as already described above. The specific antibody-peptide complex can thereby be detected easily and also rapidly by means of an enzymatic colour reaction.

A further diagnostic useable according to the invention comprises a probe, preferably a DNA probe, and/or primer. This opens up a further possibility of obtaining the nucleic acids used according to the invention, for example by isolation from a suitable gene bank, for example from a wound-specific gene bank, with the aid of a suitable probe (see, for example, J. Sambrook et al., 1989, Molecular Cloning. A Laboratory Manual 2nd edn., Cold Spring Harbor Laboratory, Cold Spring Harbor, N.Y. Chapter 8 page 8.1 to 8.81, Chapter 9 page 9.47 to 9.58 and Chapter 10 page 10.1 to 10.67).

Suitable probes are, for example, DNA or RNA fragments having a length of about 100-1000 nucleotides, preferably having a length of about 200-500 nucleotides, in particular having a length of about 300-400 nucleotides, whose sequence can be derived from the polypeptides used according to SEQ ID No. 1 to SEQ ID No. 10 of the sequence protocol and/or with the aid of the cDNA sequences of the database entries indicated in FIG. 5 (see also Example 3, 5, 6).

Alternatively, it is possible with the aid of the derived nucleic acid sequences to synthesize oligonucleotides which are suitable as primers for a polymerase chain reaction. Using this, the nucleic acid used according to the invention or parts of this can be amplified and isolated from cDNA, for example wound-specific cDNA (Examples 2, 5, 6). Suitable primers are, for example, DNA fragments having a length of about 10-100 nucleotides, preferably having a length of about 15 to 50 nucleotides, in particular having a length of 20-30 nucleotides, whose sequence can be derived from the polypeptides according to SEQ ID No. 1 to SEQ ID No. 10 of the sequence protocol and/or with the aid of the cDNA sequences of the database entries indicated in FIG. 5 (Examples 2, 5, 6).

The invention furthermore relates to a test useable according to the invention for the identification of functional interactors in connection with disorders of skin or intestinal disorders and/or treatment in wound healing and/or disorders of wound healing, which comprises at least one nucleic acid, at least one polypeptide or at least one antibody used according to the present invention, if appropriate together with suitable additives and auxiliaries.

The term “functional interactors” in the meaning of the present invention is understood as meaning all those molecules, compounds and/or compositions and substance mixtures which can interact under suitable conditions with the nucleic acids, polypeptides or antibodies used according to the invention, if appropriate together with suitable additives and/or auxiliaries. Possible interactors are simple chemical organic or inorganic molecules or compounds, but can also include nucleic acids, peptides, proteins or complexes thereof. On account of their interaction, the functional interactors can influence the function(s) of the nucleic acids, polypeptides or antibodies in vivo or in vitro or alternatively only bind to the nucleic acids, polypeptides or antibodies used according to the invention or enter into other interactions of covalent or non-covalent manner with them.

A suitable test system useable according to the invention can be produced, for example, by the stable transfection of epidermal or dermal cells with expression vectors which contain selectable marker genes and the nucleic acids used according to the invention. In this process, the expression of the nucleic acids used according to the invention is altered in the cells such that it corresponds to the pathologically disturbed expression in vivo. Anti-sense oligonucleotides which contain the nucleic acid used according to the invention can also be employed for this purpose. It is therefore of particular advantage for these systems to know the expression behaviour of the genes in disturbed regenerative processes, such as disclosed in this application. Often, the pathological behaviour of the cells in vitro can thus be mimicked and substances can be sought which reproduce the normal behavior of the cells and which have a therapeutic potential.

Suitable cells for these test systems useable according to the invention are, for example, HaCaT cells, which are generally obtainable, and the expression vector pCMV4 (Anderson et al., 1989, J. Biol. Chem. 264: 8222-9). The nucleic acid used according to the invention can in this case be integrated into the expression vectors both in the sense and in the anti-sense orientation, such that the functional concentration of mRNA of the corresponding genes in the cells is either increased, or is decreased by hybridization with the antisense RNA. After the transfection and selection of stable transformants, the cells in culture in general show an altered proliferation, migration and/or differentiation behavior in comparison with control cells. This behavior in vitro is often correlated with the function of the corresponding genes in regenerative processes in the body (Yu et al., 1997, Arch. Dermatol. Res. 289: 352-9; Mils et al., 1997, Oncogene 14: 15555-61; Charvat et al., 1998, Exp Dermatol 7: 184-90; Mythily et al., 1999, J. Gen. Virol. 80: 1707-13; Werner, 1998, Cytokine Growth Factor Rev. 9: 153-65) and can be detected using tests which are simple and rapid to carry out, such that test systems for pharmacologically active substances based thereon can be constructed. Thus, the proliferation behavior of cells can be detected very rapidly by, for example, the incorporation of labelled nucleotides into the DNA of the cells (see, for example, Fries and Mitsuhashi, 1995, J. Clin. Lab. Anal. 9: 89-95; Perros and Weightman, 1991, Cell Prolif. 24: 517-23; Savino and Dardenne, 1985, J. Immunol. Methods 85: 221-6), by staining the cells with specific stains (Schulz et al., 1994, J. Immunol. Methods 167: 1-13) or by means of immunological processes (Frahm et al., 1998, J. Immunol. Methods 211: 43-50). The migration can be detected simply by the migration index test (Charvat et al., supra) and comparable test systems (Benestad et al., 1987, Cell Tissue Kinet. 20: 109-19, Junger et al., 1993, J. Immunol. Methods 160: 73-9). Suitable differentiation markers are, for example, keratin 6, 10 and 14 and also loricrin and involucrin (Rosenthal et al., 1992, J. Invest. Dermatol. 98: 343-50), whose expression can be easily detected, for example, by means of generally obtainable antibodies.

Another suitable test system is based on the identification of functional interactions using the so-called two-hybrid system (Fields and Sternglanz, 1994, Trends in Genetics, 10, 286-292; Colas and Brent, 1998 TIBTECH, 16, 355-363). In this test, cells are transformed using expression vectors which express fusion proteins from the polypeptide used according to the invention and a DNA binding domain of a transcription factor such as, for example, Gal4 or LexA. The transformed cells additionally contain a reporter gene, whose promoter contains binding sites for the corresponding DNA- binding domains. By transformation of a further expression vector which expresses a second fusion protein from a known or unknown polypeptide having an activation domain, for example of Gal4 or herpesvirus VP16, the expression of the reporter gene can be greatly increased if the second fusion protein interacts functionally with the polypeptide used according to the invention. This increase in expression can be utilized in order to identify novel interactors, for example by preparing a cDNA library from regenerating tissue for the construction of the second fusion protein. Moreover, this test system can be utilized for the screening of substances which inhibit an interaction between the polypeptide used according to the invention and a functional interactor. Such substances decrease the expression of the reporter gene in cells which express fusion proteins of the polypeptide used according to the invention and of the interactor (Vidal and Endoh, 1999, Trends in Biotechnology; 17: 374-81). Novel active compounds which can be employed for the therapy of disorders of regenerative processes can thus be rapidly identified.

Functional interactors of the polypeptides used according to the invention can also be nucleic acids which are isolated by means of selection processes, such as, for example, SELEX (see Jayasena, 1999, Clin. Chem. 45: 1628-50; Klug and Famulok, 1994, M. Mol. Biol. Rep. 20: 97-107; Toole et al., 1996, U.S. Pat. No. 5,582,981). In the SELEX process, typically those molecules which bind to a polypeptide with high affinity (aptamers) are isolated by repeated amplification and selection from a large pool of different, single-stranded RNA molecules. Aptamers can also be synthesized and selected in their enantiomorphic form, for example as the L-ribonucleotide (Nolte et al., 1996, Nat. Biotechnol. 14: 1116-9; Klussmann et al., 1996, Nat. Biotechnol. 14: 1112-5). Thus isolated forms have the advantage that they are not degraded by naturally occurring ribonucleases and therefore have greater stability.

The invention further relates to the use of an array immobilized on a support material for analysis in connection with disorders of skin or intestine and/or wound healing and/or disorders of wound healing, which is characterized in that it comprises at least one nucleic acid and/or at least one polypeptide and/or at least one antibody useable according to the present invention.

Processes for preparing such arrays are known, for example, from WO 89/109077, WO 90/15070, WO 95/35505 and U.S. Pat. No. 5,744,305 by means of spotting, solid-phase chemistry and photolabile protective groups.

The invention further comprises the use of a DNA chip and/or protein chip for analysis in connection with disorders, in particular skin or intestinal disorders and/or wound healing and/or disorders in wound healing, which comprises at least one nucleic acid and/or at least one polypeptide and/or at least one antibody used according to the present invention. DNA chips are known, for example, from U.S. Pat. No. 5,837,832.

The invention will now be further illustrated below with the aid of the figures and examples, without the invention being restricted hereto.

### DESCRIPTION OF THE TABLES, FIGURES AND SEQUENCES

FIG. 1: Autoradiogram of hybridizations of membranes (mouse ATLAS Array, Clontech) with an identical pattern of applied cDNA fragments using four different probes. The cDNA fragments were all derived from a wound-specific, subtractive cDNA library which was enriched for those cDNAs which were expressed in the wound tissue more strongly in comparison with intact skin. All probes were prepared from cDNAs which originated from subtractive hybridizations. A: wound-specific probe (subtraction wound versus intact skin), B: skin-specific probe (subtraction intact skin versus wound), C: probe specific for badly healing wounds (subtraction wound dexamethasone-treated animals versus wound control animals), D: probe specific for normally healing wounds (subtraction wound control animals versus wound dexamethason-treated animals). The positions of the NM23-M2 cDNA (each loaded twice) are indicated with arrows.

FIG. 2: Results of the quantitative “real time RT-PCR” of NM23-M2 at different stages of wound healing in the mouse. The formula for the calculation of the abundance relative to GAPDH is indicated. The induction results from the normalization of the abundance with the abundance in intact skin.

FIG. 3: Results of the RNase protection assays of NM23-H1 with skin samples of psoriasis patients and control persons. The radioactive hybridization probe without RNase treatment (lanes 1 and 5) as well as the negative control (tRNA, lanes 2 and 6), the RNA from skin biopsies of 4 different control persons (lanes 3, 7, 8 and 9), and the RNA from skin biopsies of 3 different psoriasis patients (lanes 4, 10 and 11) each after hybridization with the probe and RNase treatment were loaded. The arrows indicate the position of the RNA-fragment of the probe which was protected against RNase degradation after hybridization with the NM23-H1 mRNA probe.

FIG. 4: Results of the RNase protection assays of NM23-H1 with intestinal samples of Crohn's disease patients and control persons. The radioactive hybridization probe without RNase treatment (lane 1) as well as the negative control (tRNA, lane 2), the RNA from a intestine biopsy of a control patient (lane 3), the RNA from intestine biopsies of Crohn's disease patients with less inflamed areas (lanes 4 and 6) and markedly inflamed areas (lanes 5, 7 and 8), each after hybridization with the probe and RNase treatment, were loaded. The RNA used in lanes 4, 5 and 6 as well as in lanes 7 and 8 each originate from the same patient. The arrow indicates the position of the RNA-fragment of the probe which was protected against RNase degradation after hybridization with the NM23-H1 mRNA probe.

FIG. 5: Tabular survey of the polypeptide sequences of the gene family NM23 identified in the analysis of gene expression during the wound-healing process and their cDNAs and accession numbers.

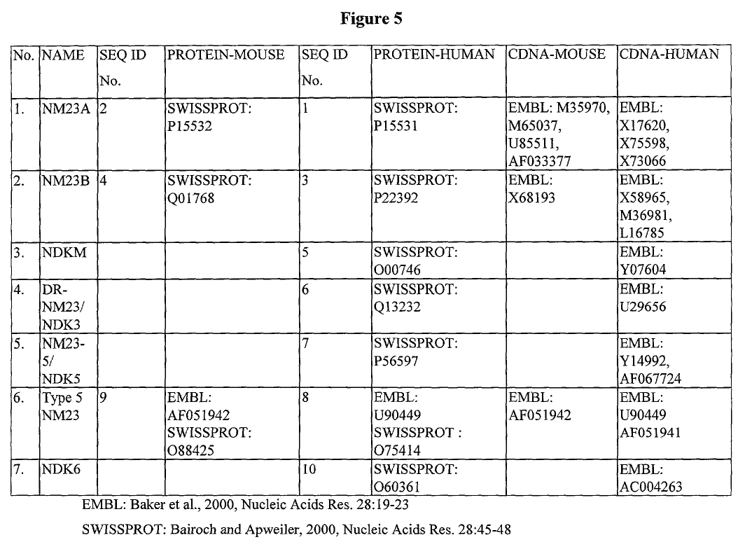

FIG. 6: Comparison of the polypeptide sequences of the identified proteins of NM23A (NDKA_human, NDKA_mouse) and NM23B (NDKB_human, NDKB_mouse) from human and mouse. Differences to the human sequence of NM23A are indicated.

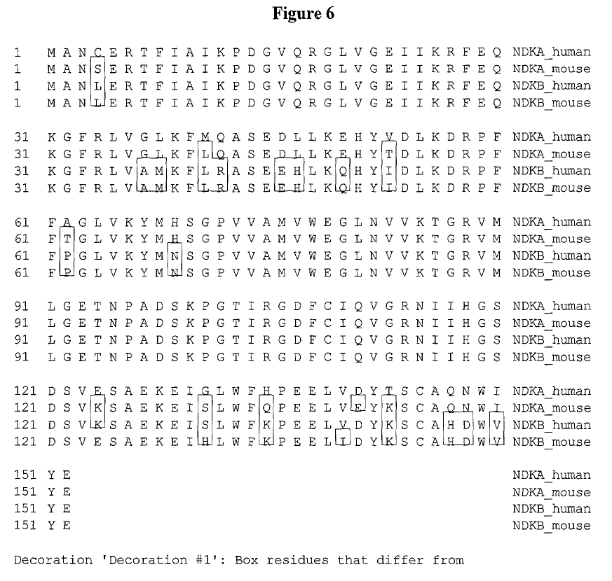

FIG. 7: Tabular survey of the amount of wound-relevant NM23-M1 and NM23-M2 cDNA at different points of time after wounding of adult mice relative to the amount of cDNA of intact skin determined by “TaqMan Assay”.

FIG. 8: Tabular survey of the amount of human wound-relevant mRNA in day-1 and day-5 wounds determined by “TaqMan Assay”.

SEQ ID No. 1 to SEQ ID No. 10 show the poly-peptides used according to the invention from human or mouse.

SEQ ID No. 11 to SEQ ID No. 14 and SEQ ID No. 17 to SEQ ID No. 26 show DNA sequences of oligonucleotides which were used for the experiments of the present invention.

SEQ ID No. 15 to SEQ ID No. 16 show DNA sequences of NM23 which were used for the preparation of probes for the RNase protection assay and in situ hybridization.

### EXAMPLES

### Example 1

### Enrichment of Wound-Relevant cDNA by Means of Subtractive Hybridization and Identification of NM23-M1 as Wound-Relevant Gene

Total RNA was isolated from intact skin and from wound tissue (wounding on the back 1 day before tissue sampling by scissors cut) of BALB/c mice by standard methods (Chomczynski and Sacchi, 1987, Anal. Biochem. 162: 156-159, Chomczynski and Mackey, 1995, Anal. Biochem. 225: 163-164). In order to obtain tissue of mice with poorly healing wounds, BALB/c mice were treated before wounding with dexamethasone (injection of 0.5 mg of dexamethasone in isotonic saline solution per kg of body weight twice per day for 5 days). The RNAs were then transcribed into cDNA with the aid of a reverse transcriptase. The cDNA synthesis was carried out using the “SMART PCR cDNA synthesis kit” from Clontech Laboratories GmbH, Heidelberg, according to the directions of the corresponding manual.

In order to identify those cDNAs which occurred with differing frequency in the cDNA pools, a subtractive hybridization (Diatchenko et al., 1996, Proc. Natl. Acad. Sci. USA 93: 6025-30) was carried out. This was effected using the “PCR select cDNA subtraction kit” from Clontech Laboratories GmbH, Heidelberg, according to the directions of the corresponding manual, the removal of excess oligonucleotides after the cDNA synthesis being carried out by means of agarose gel electrophoresis. Four cDNA pools were set up, which were enriched for wound-relevant genes, where one pool was enriched for cDNA fragments which are expressed more strongly in the wound tissue in comparison with intact skin (“wound-specific cDNA pool”), one pool was enriched in cDNA fragments which are more strongly expressed in intact skin in comparison with wound tissue (“skin-specific cDNA pool”), one pool was enriched in cDNA fragments which are more strongly expressed in normally healing wounds in comparison with poorly healing wounds (“normally healing cDNA pool”) and one pool was enriched in cDNA fragments which are more strongly expressed in badly healing wounds in comparison with normally healing wounds (“badly healing cDNA pool”).

In order to identify those genes which were contained in the cDNA pools relevant to wound healing, the presence of the corresponding cDNAs in the pools was analyzed by “reverse Northern blot”. Here, the cDNA fragments are immobilized on membranes in the form of arrays of many different cDNAs, and hybridized with a complex mixture of radio-labelled cDNA (Sambrook et al., 1989, Molecular Cloning: A Laboratory Manual, Cold Spring Harbor, Cold Spring Harbor Laboratory Press, New York, Chapter 9 page 9.47 to 9.58 and Chapter 10 page 10.38 to 10.50; Anderson and Young: Quantitative filter hybridization; in: Nucleic Acids Hybridization, A Practical Approach, 1985, Eds. Hames and Higgins, IRL Press Ltd.; Oxford, Chapter 4, page 73 to 112).

For the preparation of suitable hybridization probes, the subtracted cDNA pools were treated with the restriction endonuclease RsaI and purified by means of agarose gel electrophoresis (Sambrook et al., supra, Chapter 6, page 6.1 to 6.35) in order to separate the cDNA synthesis and amplification primer (see manual “PCR-Select cDNA Subtraction Kit”, Clontech). The cDNAs were then radio-labelled using the “random hexamer priming” method (Feinberg and Vogelstein, 1983, Anal. Biochem. 132: 6-13) in order to prepare hybridization probes.

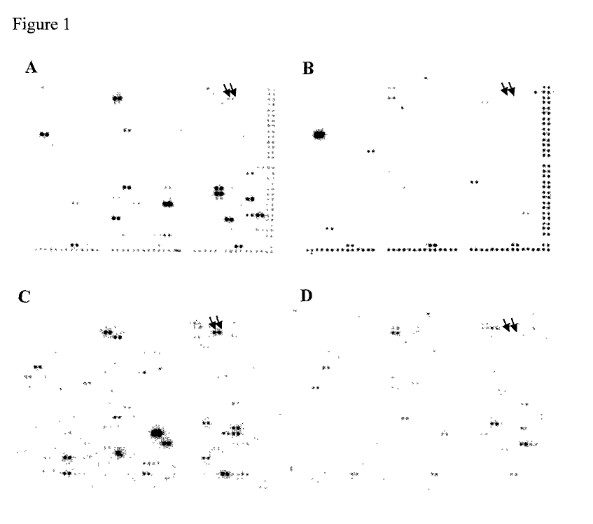

The membrane was preincubated in 25 ml of hybridization solution for 30 min at 65° C. (25 mM sodium phosphate, pH=7.5, 125 mM NaCl, 7% SDS). The hybridization probe was denatured at 100° C. for 10 min, then cooled on ice, about 100 CPM per ml were added to the hybridization solution and the hybridization was carried out in a hybridization oven for 16 hours at 65° C. The membrane was then washed twice with the hybridization solution without probe at 65° C. for 10 min. The membrane was then washed at 65° C. several times for 10 min with wash solution (2.5 mM sodium phosphate, pH=7.5, 12.5 mM NaCl, 0.7% SDS) until it was no longer possible to detect any activity in the wash solution poured off. The radioactive signals were analyzed using a phosphoimager (BioRad, Quantity One®) (FIG. 1). Those cDNAs were then selected which produced different signal intensities with the various probes. This resulted at the position of NM23-M2 on the membrane, in a slightly stronger signal intensity with the hybridization probe of the wound specific cDNA pool in comparison to the skin specific cDNA pool and a clearly stronger signal intensity with the hybridization probe of the poorly healing cDNA pool in comparison to the normally healing cDNA pool.

### Example 2

### Confirmation of the Expression Pattern of NM23-M2 by Means of “Real-Time Quantitative RT-PCR”

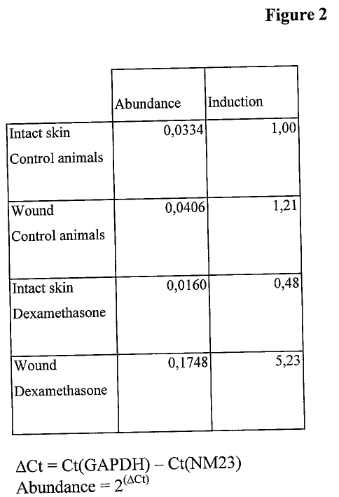

A confirmation of the differential expression of the nucleic acids used according to the invention was carried out by real-time RT-PCR in the ABI Prism 7700 sequence detection system (PE Applied Biosystems). The apparatus was equipped with the ABI Prism 7200/7700 SDS software version 1.6.3 (1998). The detection of PCR products was carried out during the amplification of the cDNA with the aid of the stain SYBR Green 1, whose fluorescence is greatly increased by binding to double-stranded DNA (Karlsen et al. 1995, J. Virol. Methods. 55: 153-6; Wittwer et al., 1997, BioTechniques 22: 130-8, Morrison et al., 1998, BioTechniques 24: 954-62). The basis for the quantification is the PCR cycle (threshold cycle, C_{T}-value) which is reached when the fluorescence signal exceeds a defined threshold. The analysis is carried out by means of the Δ-C_{T} method (User Bulletin #2, Relative Quantification of Gene Expression, PE Applied Biosystems, 1997). The abundance of the cDNAs were determined relative to an endogenous reference (GAPDH). The results are shown in FIG. 2.

Total RNA pools from skin and wound tissue from 16 animals each was obtained as described above and 1 μg of total RNA was subjected to reverse transcription in a thermocycler (GeneAmp PCR system 9700, PE) using the TaqMan reverse transcription reagent kit (PE) according to the recommendations of the manufacturer (SYBR Green PCR and RT-PCR Reagents Protocol, PE Applied Biosystems, 1998). The primers for the amplification of the NM23-M2 cDNA (NM23-Primer 1: TTCAAAACCAGGCACCATCC (SEQ ID No. 11), NM23-Primer 2: ACTCTCCACTGAATCACTGCCA (SEQ ID No. 12) and the reference (GAPDH primer 1: ATCAACGGGAAGCCCATCA (SEQ ID No. 13), GAPDH primer 2: GACATACTCAGCACCGGCCT (SEQ ID No. 14)) were selected with the aid of the Primer Express software for Macintosh PC Version 1.0 (PE Applied Biosystems, P/N 402089, 1998) based on the nucleic acid used according to the invention and the known sequence of GAPDH. For the PCR, the SYBR Green PCR Core Reagents Kit (4304886, PE Applied Biosystems) was used. The concentration of the primers in the PCR was initially optimized in the range from 50 nM to 600 nM and the specificity of the PCR was verified by analysis of the length of the amplified products by agarose gel electrophoresis. The efficiency of the PCR system was then determined by means of a dilution series (User Bulletin #2, Relative Quantification of Gene Expression, PE Applied Biosystems, 1997). It became apparent that for both cDNAs the efficiency of the amplification was 100%, i.e. at each 1:2 dilution of the cDNA one more cycle was needed in order to exceed the fluorescence threshold value.

For the quantification, each batch of cDNA was amplified from 10 ng of reverse-transcribed total RNA in a total volume of 25 μl. The running conditions for the PCR corresponded to the details of the manufacturer (PE Applied Biosystems, SYBR Green® PCR and RT-PCR Reagents Protocol, 1998). The C_{T}-values were analyzed and the abundance of NM23-M2 relative to GAPDH was calculated. It was possible to confirm the slight induction of NM23-M2 in normally healing wounds and the strong induction in poorly healing wounds of dexamethasone-treated animals (FIG. 2).

### Example 3

### Verification of the Expression Pattern of NM23-H1 by Means of “RNase Protection Assays”

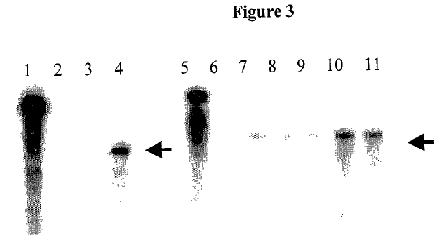

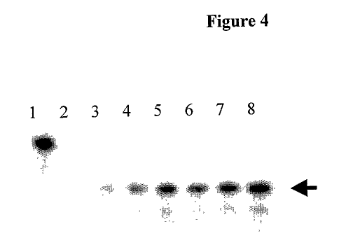

The expression of NM23-H1 was verified with the aid of the “RNase protection assay”. The test was carried out as described in the literature (Sambrook et al., supra Chapter 7, page 7.71 to 7.78; Werner et al., 1992; Growth Factors and Receptors: A Practical Approach 175-197, Werner, 1998, Proc. Natl. Acad. Sci. USA 89: 6896-6900). Reverse-strand RNA which was transcribed in vitro and radio-labelled was used as a hybridization probe. A NM23-H1 fragment of 266 bp length (SEQ. ID No. 15) was cloned via blunt ends into the EcoRV-restriction site of the vector pBluescript II KS (Stratagene). The plasmid was linearized with XbaI before transcription (length of the transcript without vector sequence: 299 basepairs, sequence of the probe SEQ ID No. 15). The transcriptions were carried out with T3 polymerase (Roche Diagnostics, Mannheim) in the presence of ³²P-UTP (35 μCi/batch) (Amersham, Brunswick) according to the details of the manufacturer. The probe was purified by gel electrophoresis and elution (Sambrook et al., supra, Kapitel 6, Seite 6.36 bis 6.48). For the hybridization reaction, about 10⁵ CPM each of the labelled transcripts were employed. For this, 10 μg of total RNA which was isolated from either skin or intestine biopsies using standard methods (Chomczynski and Sacchi, 1987, Anal. Biochem. 162: 156-159, Chomczynski and Mackey, 1995, Anal. Biochem. 225: 163-164) was precipitated together with the transcript, taken up in 10 μl of hybridization buffer (80% deionized formamide, 400 mM NaCl, 40 mM Pipes pH 4.6, 1 mM EDTA) and hybridized overnight at 42° C. An RNase A/T1 digestion (Boehringer, RNase A: 0.8 μg/batch, RNase T1: 20 U/batch) was then carried out. After inactivation of the RNase by proteinase K digestion (Boehringer, 30 μg/batch) and phenol extraction, the samples were precipitated with ethanol according to standard methods (Sambrook et al., supra). The samples were then separated by gel electrophoresis on a denaturing 5% acrylamide gel (7_M urea) . The gel was dried and the radioactive signals were analyzed by means of autoradiography (FIGS. 3 and 4).

In the RNase protection assay with RNA isolated from biopsies of 3 different psoriasis and 4 different control patients, an increased expression of NM23-H1 could be observed in the skin samples from all psoriasis patients in comparison to the control skin samples (FIG. 3). The RNase protection assay with RNA from tissue samples derived from 5 persons with Crohn's disease showed an increased expression of NM23-H1 in inflammatory slices of the intestine relative to control subjects (FIG. 4).

### Example 4

### Analysis of the Expression of NM23-H1 in Tissue Cuts of Mouse Wounds

The expression of NM23-M1 in wounds was analyzed by means of in situ hybridization. The test was performed as described in the literature (Werner et al., 1992; Growth Factors and Receptors: A Practical Approach 175-197, Werner, 1998, Proc. Natl. Acad. Sci. USA 89: 6896-6900). A NM23-M1 fragment of 256 bp length (SEQ. ID No. 16) was cloned via blunt ends into the EcoRV-restriction site of the vector pBluescript II KS (Stratagene). Using this vector, a radiolabelled reverse-strand RNA as hybridization probe was produced and used as described (Werner, 1998, supra). This probe was used to perform hybridizations on frozen tissue slices of 5 day-wounds of mice. It became apparent that the NM23-M1 gene was increasingly expressed in the hyperproliferative epithelium at the woundedge. In contrast, in healthy parts of the skin, a faint to nondetectable staining was observed.

### Example 5

### Analysis of Murine Patterns of Expression of NM23-M1 and NM23-M2 mRNAs During Wound Healing by Means of “TaqMan Analysis”

The kinetics of regulation of expression of NM23 mRNA's NM23-M1 and NM23-M2 during normal wound healing of the adult mouse was analyzed using “TaqMan Analysis” in GeneAmp5700 of Applied Biosystems. Normally healing wounds and intact skin were taken from 10 week old BALB/c mice using scissors cut as described above.

In order to isolate RNA, the biopsies were homogenized in the presence of RNAclean buffer (AGS, Heidelberg) that was supplemented by 1/100 volume fraction of 2-mercapto-ethanol using a disperser. Subsequently the RNA was extracted by a twice repeated phenolization using water saturated acidic phenol in the presence of 1-bromo-3-chloro-propane. Then the RNA was precipitated using isopropanol and ethanol precipitation and the RNA was washed using 75% ethanol. Afterwards the RNA was treated with DNaseI. 20 μg of RNA (ad 50 μl DEPC-treated water) was supplemented with 5.7 μl transcription buffer (Roche), 1 μl RNase-inhibitor (Roche); 40 U/μl) and 1 μl DNaseI (Roche); 10 U/μl) and incubated for 20 minutes at 37° C. Then another 1 μl DNaseI was added and the sample was incubated for another 20 minutes at 37° C. Subsequently the RNA was phenolized, ethanol precipitated and washed. All above mentioned steps were carried out using DEPC(diethylpyrocarbonate)-treated solutions and liquids unless they contained reactive aminogroups. Subsequently cDNA was synthesized from the extracted RNA. 20 μl RNA (50 ng/μl) were supplemented with 1× TaqMan RT-buffer (Perkin Elmer), 5.5 mM MgCl₂ (Perkin Elmer), 500 μM dNTPs each (Perkin Elmer), 2.5 μM random hexameres (Perkin Elmer), 1.25 μl Multiscribe Reverse Transcriptase (50 U/μl Perkin Elmer), 0,4 μl RNase-inhibitor (20 U/μl, Perkin Elmer) and DEPC-treated water (ad 100 μl volume).

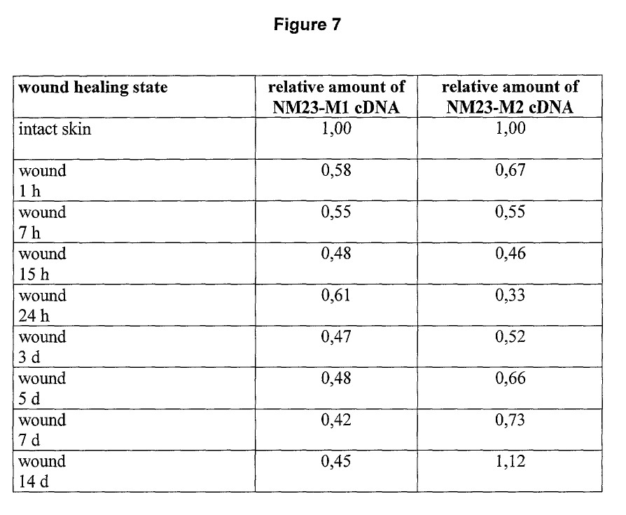

Upon addition of RNA and thorough mixing the solutions were divided into two 0.2 ml tubes (50 μl each) and the reverse transcription reaction was carried out in a thermocycler (10 min at 25° C.; 30 min at 48° C. and 5 min at 95° C.). The following quantification of cDNA was done by means of quantitative PCR using the SYBR Green PCR Master Mixes (Perkin Elmer), wherein for each NM23 cDNA species to be quantified, a triple determination (each time with target primers and GAPDH primers) was carried out. The stock solution for each triplet contained at a total volume of 57 μl, 37.5 μl 2× SYBR Master Mix, 0.75 μl Amp. Erase UNG(1 U/μl) and 18.75 μl DEPC-treated water. For each triple determination the 57 μl stock solution were supplemented with 1.5 μl forward- and reverse primer each in a concentration ratio that had been optimized before. Each 60 μl stock solution/primer mix were mixed with 15 μl cDNA solution (2 ng/μl) and divided to 3 wells. In parallel a stock solution with primers for the determination of GAPDH (SEQ ID No. 13 and SEQ ID No. 14) as a reference as mixed with additional 15 μl of the same cDNA solution and distributed onto 3 wells. In order to obtain a standard graph for the GAPDH-PCR, a dilution series of different cDNA solutions was made (4 ng/μl; 2 ng/μl; 1 ng/μl; 0.5 ng/μl and 0.25 ng/μl). For the determination of GAPDH,15 μl each of the CDNA solutions of the dilution series was mixed with 60 μl stock solution/primer mix and distributed onto 3 wells. A standard graph for each of the PCRs of the NM23 homologues to be analyzed was obtained; wherein the same dilutions used for the GAPDH standard graph were used. As a control a PCR without cDNA was used. The stock solution/primer mix of each the target and GAPDH were supplemented with 15 μl DEPC-water, mixed and distributed onto 3 wells. The amplification was performed using Gene Amp. 5700 (2 min at 50° C.; 10 min at 95° C., followed by 3 cycles with 15 s at 96° C. and 2 min at 60° C.; followed by 37 cycles with 15 s at 95° C. and 1 min at 60° C.) . The analysis was done by determining the abundance for each NM23 gene relative to the GAPDH-reference. The standard curve was determined first by plotting the C_{T}-values of the dilution series against the logarithm of the amount of cDNA and the PCR (ng of transcribed RNA) and the slope of the graph was determined. The efficiency (E) of the PCR can be calculated as follows: E=10^{-1/s}−1. The relative abundance (X) of the NM23 cDNA species (Y) under investigation with respect to GAPDH is: X=(1+E_{GAPDH})_{T}^{C (GAPDH)}/(1+E_{Y}) _{T}^{C (Y)}. Subsequently the values were standardized by equatizing the amount of cDNA in intact skin of adult 10 weeks old BALB/c control animals with 1. The relative changes of expression of NM23-M1 and NM23-M2 respectively at different points of time after wounding of adult mice are depicted in FIG. 7.

Using appropriate primers to detect NM23-M1 (NM23-primer 3: TCC TGG CAC AGT CAG ACA ACA (SEQ ID No. 17); NM23-primer 4: TTC ACA ACC TCA CAC ATC CTC C (SEQ ID No. 18)) and NM23-M2 (NM23-primer 1: (SEQ ID No. 11) and NM23-primer 2 (SEQ ID No. 12)), it could be shown that the expression of both homologues was reduced during wound healing in adult mice (FIG. 7).

In the course of the wound healing NM23-M1 expression was overall constantly reduced to about 50% of the amount observed in intact skin. A similar pattern was detected in NM23-M2 expression, where during wound healing between 1 h and 7 d after wounding the expression was reduced considerably relative to the intact skin. Only after d 14 after wounding the expression level rebounded to approximately the original level. Taken together both homologues showed a comparably reduced level of expression over a long time of the wound healing. This result seems to contradict the examples 1 and 2, where a weak increase in the amount of NM23 expression in normal healing wounds could be observed. However, in example 4 it was demonstrated, that there is no overall increase in NM23 expression in wound tissue slices. An increased staining was observed in the hyperproliferative epithelium, but no significant expression was detected in other layers of the tissue. This result implies that wound healing-dependent changes in the amount of mRNA during complex changes of the spatial pattern of expression can only be analyzed in an insufficient way since the sensitivity to detect mRNA is lower in the case of in situ hybridization than in the case of “real time RT-PCR”: due to the complex changes in the spatial patterns of NM23 expression, subtle variabilities during the taking of the biopsies may lead to different results concerning wound healing dependent changes of the amount of NM23 mRNA determined by means of “real time RT-PCR” (example 2) and subtractive hybridization (example 1) as opposed to a determination by means of “TaqMan analysis” (example 5). It was surprising that genes of the NM23 gene family could be identified despite of the difficult conditions.

### Example 6

### Differential Expression of NM23-H1 and NM23-H2 in Human Wounds

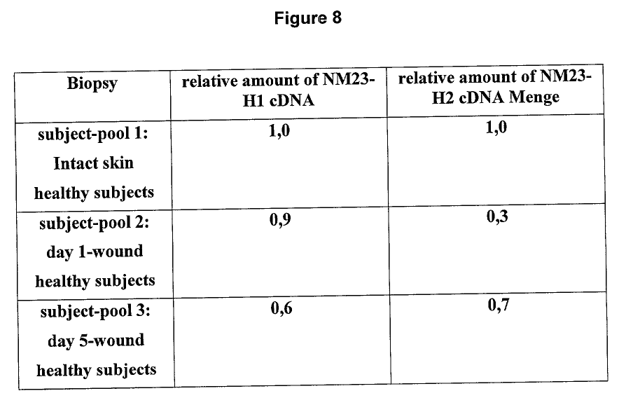

The differential regulation of NM23-H1 in psoriasis and Crohn's disease was analyzed in example 3. Using normally healing wounds it was investigated whether the differential regulation of expression of NM23-M1 and NM23-M2 shown in example 5 can also be observed in humans. Skin samples were taken from untreated intact skin, day-1 wounds or day-5 wounds of healthy subjects by means of isolating 4 mm or 6 mm punch skin samples respectively. For each group (intact skin, day-1 wound, day-5 wound) biopsies of 14 subjects each were pooled. The biopsies were desintegrated in a swing mill and the RNA was isolated as described in example 5, then DNAseI digested and reverse transcribed into cDNA. A quantification of wound healing relevant cDNA was performed as described in example 5. The results of the experiments are depicted in FIG. 8. For the analysis of NM23-homologues primers for the amplification (hGAPDH-Primer 1: CATGGGTGTGAACCATGAGAAG (SEQ ID Nr. 25); hGAPDH-Primer 2: GCTAAGCAGTTGGTGGTGCA (SEQ ID Nr. 26), NM23-H1 Primer 1: GAAATTCATGCAAGCTTCCGA (SEQ ID Nr. 19), NM23-H1 Primer 2: CAGGTCAACGTAGTGTTCCTTGAG (SEQ ID Nr. 20); NM23-H2 Primer 1: CTGGTTGACTACAAGTCTTGTGCTC (SEQ ID Nr. 21); NM23-H2 Primer 2: TCCACCTCTTATTCATAGACCCAGT (SEQ ID Nr. 22) were selected based on known sequences of human GAPDH (GenBank:M17851) and human NM23-H1 and NM23-H2 (EMBL:X17620 and EMBL:X58965) . cDNA resulting from reverse transcription of 10 ng total RNA was amplified in a total volume of 25 μl for quantification. PCR was performed according to the instructions of the manufacturer (PE Applied Biosystems, SYBR Green PCR and RT-PCR reagents protocol, 1998). C_{T}-values were determined and the abundance of NM23 mRNA relative to GAPDH-mRNA was calculated. The results of the experiment are depicted in FIG. 8. It was observed that wound tissue showed a slight decrease of expression. In contrast to the analysis of murine biopsies of example 5 two different points of time were selected. Using human tissue a significant coincidence of the results regarding the kinetics of wound healing of mice and of humans was observed (example 5): compared to the initial value, the NM23-M1 and NM23-H1 expression level in biopsies at day 1 after wounding is reduced by 70% and both mRNA levels show a slight increase to 66% or 70% of the initial value respectively 5 days after wounding. The weaker level of expression of NM23-M2 and NM23-H2 in day-5 wounds is also comparable (48% vs. 60% of the initial value).

Thus it could be confirmed that both genes play an essential role in the regulation of wound healing and that the modulation of the amount of at least one homologue, preferentially both homologues can be used for the prevention and/or diagnosis and/or treatment of disorders of skin cells.

### Example 7

### Lokalization of NM23-H2 in Biopsies of Intact Skin, of Normally Healing Wounds, Ulcer, As Well As Non-Lesional and Lesional Psoriatic Skin by Means of in Situ Hybridization

For the experiment biopsies of healthy skin as well as normally healing day-5 wounds were taken from a healthy subject as described in example 6. In addition biopsies of non-lesional and lesional skin of 10 psoriasis patients each and from intact skin and the wound of an ulcer patient (Ulcus cruris venosum) were taken as described above. The tissue slices were fixed in 4% paraformaldehyd, treated with proteinase K (1 μg/ml isotonic saline) for 10 min at 37° C., and subsequently treated with paraformaldehyde and then with acetanhydride (0.5 ml in 0.1 M triethanolamine, pH 8,0).

The mRNA of human NM23-H2 was localized by radioactive in situ hybridization. Paraformaldehyde fixed slices were embedded in paraffin. The synthesis of the hybridization probe was based on in vitro transcription of a partial NM23-H2 cDNA fragment in the presence of α-³⁵S-UTP. In order to obtain the PCR product promoter sequences for the transcription in sense and anti-sense direction were added to the primers. (T3-NM23-H2-primer: AATTAACCCTCACTAAAGGGGGAGGGGCTGAACGTGGTGAAGAC (sense control primers with T3-promoter; SEQ ID No. 23), Sp6-NM23-primer: ATTTAGGTGACACTATAGAATACACGCCGTGCTGAAGGAGACTGC (antisense primer with Sp6-promoter; SEQ ID No. 24). For the in vitro transcription 60 μCi ³⁵S-UTP and 5 mM ATP, GTP and CTP each, as well as either 25 U T3- or T7-RNA polymerase (Roche), 1 μg PCR-product, 10 mM Dithiothreitol, 40 U RNAse inhibitor (Roche) and 1× TB-buffer (Roche) were used.

Human tissue slices (see above) were mounted onto slides, treated with proteinase K, fixed with para-formaldehyde and were subsequently acetylated. The slices were transferred into a humid chamber containing Whatman tissue paper soaked in 50% formamide/4× SSC. The slices were covered with 30 μl hybridization solution and incubated for 2.5 h at 60° C. Afterwards, the slices were incubated with 30 μl hybridization solution containing 0.7×10⁶ CPM of radioactively labelled riboprobe for 16 h at 60° C. Then the slices were washed under stringent conditions incubated with RNAse A and dehydrated with ethanol. The slices were then covered with photo emulsion (Kodak IBO 1433) in the absence of light and oxygen for 2-6 weeks at 40° C. and subsequently developed using photographic developer and fixative (Kodak IBO 1433) according to the instructions of the manufacturer.

No or very weak signals were observed in intact skin of the healthy subject and the ulcer-patient as well as in non-lesional skin of the psoriasis patients. In contrast, tissue slices of normal healing day-5 wounds showed signals in the basal cell layer of the hyperproliferative epithelium. This indicates that the induction of NM23-H2 expression is particularly essential in the cell layer that is responsible for the closure of the wound by means of proliferation and migration. This is consistent with the observation that no significant labelling was detected at wound edge and the wound ground of the non- or badly-healing Ulcer wound. Thus, the wound specific regulation of NM23-H2-expression is essential for the normal process in wound healing. In comparison to intact skin of the healthy subject or non-lesional skin of the psoriasis patients, the lesional psoriatic skin biopsies also showed significantly increased labelling intensity in the basal cell layers of the hyperproliferative epithelium. This is consistent with a result of the experiment of example 3 where it was shown that the amount of NM23-H1 is increased in psoriatic skin.

This experiment elucidates that the regulation of NM23 expression is essential for intact, healthy skin as well as for the normal process of wound healing and that a dysfunctional regulation of the expression can lead to disorders of skin cells for example to a delayed wound healing or psoriasis and it shows that NM23, preferentially both homologues can be used for the prevention and/or diagnosis and/or treatment of disorders of skin cells. Badly healing wounds are associated with a reduced amount of NM23, whereas in psoriasis patients, whose keratinocytes are characterised by pathological proliferation, exhibit an increase in the level of NM23 expression.

For the treatment of skin cells the expression or activity of NM23 should be modulated, preferentially by activating the expression or activity of NM23 in the case of disorders of wound healing. The activity or expression of NM23 should be preferentially inhibited in the case of hyperproliferative disorders of skin cells, especially in the case of psoriasis.

It will be apparent to those skilled in the art that various modifications can be made to the compositions and processes of this invention. Thus, it is intended that the present invention cover such modifications and variations, provided they come within the scope of the appended claims and their equivalents.

Priority application DE 10008330.7-41, filed Feb. 23, 2000 and priority application U.S. Ser. No. 60/199,312 filed Apr. 24, 2000. All publications cited herein are incorporated in their entireties by reference.

1. Method of using at least one polypeptide according to one of SEQ ID No. 1 to SEQ ID No. 10 of the gene family NM23 or a functional variant thereof or at least one nucleic acid coding for one of said polypeptides or a variant thereof, for analysis and/or diagnosis and/or prevention and/or treatment of disorders of skin and/or intestinal disorders and/or treatment in wound healing and/or disorders of wound healing.

2. Method of using at least one polypeptide according to one of SEQ ID No. 1 to SEQ ID No. 10 of the gene family NM23 or a functional variant thereof or at least one nucleic acid coding for one of said polypeptides or a variant thereof, for identification of at least one pharmacologically active substance relating to disorders of skin and/or intestinal disorders and/or treatment in wound healing and/or disorders of wound healing.

3. Method of using according to claim 2, characterized in that the pharmacologically active substance is selected from a nucleic acid, for example in the form of a DNA-binding domain, a promoter or an enhancer, a repressor, or a polypeptide, for example in the form of an activator or an inhibitor.

4. Method of using a nucleic acid according to claim 1 or 2, characterized in that the nucleic acid is a DNA or RNA, preferably a DNA, in particular a double-stranded DNA.

5. Method of using a nucleic acid according to claim 1 or 2, characterized in that the sequence of the nucleic acid has at least one intron and/or a polyA sequence.

6. Method of using a nucleic acid according to claim 1 or 2 in the form of its antisense sequence.

7. Method of using a nucleic acid according to claim 1 or 2, characterized in that the nucleic acid has been prepared synthetically.

8. Method of using a polypeptide according to claim 1 or 2, characterized in that the polypeptide has been prepared synthetically.

9. Method of using a polypeptide according to claim 1 or 2, characterized in that the polypeptide is a fusion protein.

10. Method of using a vector, preferentially in the form of a plasmid, shuttle vector, phagemid, cosmid, containing at least one nucleic acid, coding for a polypeptide according to SEQ ID No. 1 to SEQ ID No. 10 of the gene family NM23, or a variant thereof, for analysis and/or diagnosis and/or prevention and/or treatment of disorders of skin and/or intestinal disorders and/or in wound healing and/or disorders of wound healing.

11. Method of using according to claim 10, characterized in that the vector is an expression vector.

12. Method of using according to claim 10, characterized in that the vector is a knock-out gene construct.

13. Method of using according to claim 10, characterized in that the vector is a vector suitable for gene therapy.

14. Method of using a host cell containing at least one nucleic acid coding for a polypeptide according to one of SEQ ID No. 1 to SEQ ID No. 10 of the gene family NM23 or a variant thereof, for analysis and/or diagnosis and/or prevention and/or treatment of disorders of skin and/or intestinal disorders and/or in wound healing and/or disorders of wound healing.

15. Method of using a host cell according to claim 14, characterized in that the nucleic acid is inserted into the host cell in the form of a vector according to claim 10.

16. Method of using a host cell according to claim 14, characterized in that it is a skin or intestinal cell.

17. Method of using a host cell according to claim 14, characterized in that it is a transgenic embryonic non-human stem cell.

18. Method of using a transgenic non-human mammal, containing a transgenic embryonic non-human stem cell according to claim 17, characterized in that transgenic non-human mammal is used for analysis and/or diagnosis of disorders of skin and/or intestinal disorders and/or in wound healing and/or disorders of wound healing.

19. Method of using a transgenic non-human mammal according to claim 18, characterized in that its genome contains an expression cassette or a knock-out gene construct according to claim 11 or 12.

20. Method of using an antibody for analysis and/or diagnosis and/or prevention of disorders of skin and/or intestinal disorders and/or of wound healing and/or disorders of wound healing and/or for identification of pharmacologically active substances, characterized in that said antibody is directed against a polypeptide according to one of SEQ ID No. 1 to SEQ ID No. 10 of the NM23 gene family or against a functional variant thereof.

21. Method of using a diagnostic for diagnosis of disorders of skin or intestinal disorders and/or in wound healing and/or disorders of wound healing, characterized in that it contains at least one polypeptide according to one of SEQ ID No. 1 to SEQ ID No. 10 of the NM23 gene family or a functional variant thereof or at least one nucleic acid coding for these, or a variant thereof or at least one antibody according to claim 20, if appropriate with suitable additives and/or auxiliaries.

22. Method of using according to claim 21, characterized in that the diagnostic contains a probe, preferentially a DNA-probe.

23. Method of using at least one polypeptide according to one of SEQ ID No. 1 to SEQ ID No. 10 of the NM23 gene family or a functional variant thereof or at least one nucleic acid coding for said polypeptides, or a variant thereof or at least one antibody according to claim 20, if appropriate with suitable additives or auxiliaries, for the preparation of a medicament, characterized in that it is used for the treatment of disorders of skin and/or intestinal disorders and/or in wound healing and/or disorders of wound healing.

24. Method of using a test for the identification of functional interactors in connection with disorders of skin and/or intestinal disorders and/or in wound healing and/or disorders of wound healing, characterized in that it contains at least one polypeptide according to one of SEQ ID No. 1 to SEQ ID No. 10 of the NM23 gene family or a functional variant thereof or at least one nucleic acid coding for these, or a variant thereof or at least one antibody according to claim 20, if appropriate with suitable additives and/or auxiliaries.

25. Method of using an array immobilized on a support material for analysis in connection with skin and/or intestinal disorders and/or in wound healing and/or disorders of wound healing, characterized in that it contains at least one polypeptide according to one of SEQ ID No. 1 to SEQ ID No. 10 of the NM23 gene family or a functional variant thereof or at least one nucleic acid coding for said polypeptides, or a variant thereof or at least one antibody according to claim 20.

26. Method of using according to claim 25, characterized in that the array is a DNA chip and/or protein chip.


SEQUENCE LISTING (26 sequences)

SEQ ID NO: 1 - protein, 152 aa, Homo sapiens
MANCERTFIAIKPDGVQRGLVGEIIKRFEQKGFRLVGLKFMQASEDLLKEHYVDLKDRPFFAGLVKYMHSGPVVAMVWEGLNVVKTGRVMLGETNPADSKPGTIRGDFCIQVGRNIIHGSDSVESAEKEIGLWFHPEELVDYTSCAQNWIYE

SEQ ID NO: 2 - protein, 152 aa, Mus musculus
MANSERTFIAIKPDGVQRGLVGEIIKRFEQKGFRLVGLKFLQASEDLLKEHYTDLKDRPFFTGLVKYMHSGPVVAMVWEGLNVVKTGRVMLGETNPADSKPGTIRGDFCIQVGRNIIHGSDSVKSAEKEISLWFQPEELVEYKSCAQNWIYE

SEQ ID NO: 3 - protein, 152 aa, Homo sapiens
MANLERTFIAIKPDGVQRGLVGEIIKRFEQKGFRLVAMKFLRASEEHLKQHYIDLKDRPFFPGLVKYMNSGPVVAMVWEGLNVVKTGRVMLGETNPADSKPGTIRGDFCIQVGRNIIHGSDSVKSAEKEISLWFKPEELVDYKSCAHDWVYE

SEQ ID NO: 4 - protein, 152 aa, Mus musculus
MANLERTFIAIKPDGVQRGLVGEIIKRFEQKGFRLVAMKFLRASEEHLKQHYIDLKDRPFFPGLVKYMNSGPVVAMVWEGLNVVKTGRVMLGETNPADSKPGTIRGDFCIQVGRNIIHGSDSVESAEKEIHLWFKPEELIDYKSCAHDWVYE

SEQ ID NO: 5 - protein, 187 aa, Homo sapiens
MGGLFWRSALRGLRCGPRAPGPSLLVRHGSGGPSWTRERTLVAVKPDGVQRRLVGDVIQRFERRGFTLVGMKMLQAPESVLAEHYQDLRRKPFYPALIRYMSSGPVVAMVWEGYNVVRASRAMIGHTDSAEAAPGTIRGDFSVHISRNVIHASDSVEGAQREIQLWFQSSELVSWADGGQHSSIHPA

SEQ ID NO: 6 - protein, 168 aa, Homo sapiens
MICLVLTIFANLFPAACTGAHERTFLAVKPDGVQRRLVGEIVRRFERKGFKLVALKLVQSSEELLREHYAELRERPFYGRLVKYMASGPVVAMVWQGLDVVRTSRALIGATNPADAPPGTIRGDFCIEVGNLIHGSDSVESARREIALWFRADELLCWEDSAGHWLYE

SEQ ID NO: 7 - protein, 212 aa, Homo sapiens
MEISMPPPQIYVEKTLAIIKPDIVDKEEEIQDIILRSGFTIVQRRKLRLSPEQCSNFYVEKYGKMFFPNLTAYMSSGPLVAMILARHKAISYWLELLGPNNSLVAKETHPDSLRAIYGTDDLRNALHGSNDFAAAEREIRFMFPEVIVEPIPIGQAAKDYLNLHIMPTLLEGLTELCKQKPADPLIWLADWLLKNNPNKPKLCHHPIVEEPY

SEQ ID NO: 8 - protein, 194 aa, Homo sapiens
MTQNLGSEMASILRSPQALQLTLALIKPDAVAHPLILEAVHQQILSNKFLIVRMRELLWRKEDCQRFYREHEGRFFYQRLVEFMASGPIRAYILAHKDAIQLWRTLMGPTRVFRARHVAPDSIRGSFGLTDTRNTTHGSDSVVSASREIAAFFPDFSEQRWYEEEEPQLRCGPVCYSPEGGVHYVAGTGGLGPA

SEQ ID NO: 9 - protein, 189 aa, Mus musculus
MTSILRSPQALQLTLALIKPDAVAHPLILEAVHQQILSNKFLIVRTRELQWKLEDCRRFYREHEGRFFYQRLVEFMTSGPIRAYILAHKDAIQLWRTLMGPTRVFRARYIAPDSIRGSLGLTDTRNTTHGSDSVVSASREIAAFFPDFSEQRWYEEEEPQLRCGPVHYSPEEGIHCAAETGGHKQPNKT

SEQ ID NO: 10 - protein, 137 aa, Homo sapiens
MQCGLVGKIIKRFEQKGFRLVAMKFLPASEEHLKQHYIDLKDRPFFPGLVKYMNSGPVVAMVWEGLNVVKTGRVMLGETNPADSKPGTIRGDFCIQVGRNIIHGSDSVKSAEKEISLRFKPEELVDYKSCAHDWVYE

SEQ ID NO: 11 - DNA, 20 bp, Mus musculus
ttcaaaaccaggcaccatcc

SEQ ID NO: 12 - DNA, 22 bp, Mus musculus
actctccactgaatcactgcca

SEQ ID NO: 13 - DNA, 19 bp, Mus musculus
atcaacgggaagcccatca

SEQ ID NO: 14 - DNA, 20 bp, Mus musculus
gacatactcagcaccggcct

SEQ ID NO: 15 - DNA, 266 bp, Homo sapiens
tctgaaattcatgcaagcttccgaagatcttctcaaggaacactacgttgacctgaaggaccgtccattctttgccggcctggtgaaatacatgcactcagggccggtagttgccatggtctgggaggggctgaatgtggtgaagacgggccgagtcatgctcggggagaccaaccctgcagactccaagcctgggaccatccgtggagacttctgcatacaagttggcaggaacattatacatggcagtgattctgtggagagtg

SEQ ID NO: 16 - DNA, 256 bp, Mus musculus
ctgaagtttctgcaggcttcagaggaccttctcaaggagcactacactgacctgaaggaccgccccttctttactggcctggtgaaatacatgcactcaggaccagtggttgctatggtctgggagggtctgaatgtggtgaagacaggccgcgtgatgcttggagagaccaaccccgcagactctaagcctgggaccatacgaggagacttctgcatccaagttggcaggaacatcattcatggcagcgattctg

SEQ ID NO: 17 - DNA, 21 bp, Mus musculus
tcctggcacagtcagacaaca

SEQ ID NO: 18 - DNA, 22 bp, Mus musculus
ttcacaacctcacacatcctcc

SEQ ID NO: 19 - DNA, 21 bp, Homo sapiens
gaaattcatgcaagcttccga

SEQ ID NO: 20 - DNA, 24 bp, Homo sapiens
caggtcaacgtagtgttccttgag

SEQ ID NO: 21 - DNA, 25 bp, Homo sapiens
ctggttgactacaagtcttgtgctc

SEQ ID NO: 22 - DNA, 25 bp, Homo sapiens
tccacctcttattcatagacccagt

SEQ ID NO: 23 - DNA, 44 bp, Artificial Sequence
  sense NM23H2-primer with T3 promoter sequence for PCR cloning and /or the construction of a hybridization probe
aattaaccctcactaaagggggaggggctgaacgtggtgaagac

SEQ ID NO: 24 - DNA, 45 bp, Artificial Sequence
  antisense NM23-primer with SP6 promoter sequence for PCR cloning  and/or construction of a hybridization probe
atttaggtgacactatagaatacacgccgtgctgaaggagactgc

SEQ ID NO: 25 - DNA, 22 bp, Homo sapiens
catgggtgtgaaccatgagaag

SEQ ID NO: 26 - DNA, 20 bp, Homo sapiens
gctaagcagttggtggtgca